(19) United States
(12) Patent Application Publication
(10) Pub. No.: US 2024/0317797 A1
(43) Pub. Date: Sep. 26, 2024

(54) METHODS AND USES FOR RESTORING, SOLUBILIZING AND STABILIZING POLYPEPTIDES

(71) Applicant: UNIVERSITY OF PRINCE EDWARD ISLAND, Charlottetown (CA)

(72) Inventor: Marya Ahmed, Charlottetown (CA)

(21) Appl. No.: 18/571,187

(22) PCT Filed: Jun. 15, 2021
(86) PCT No.: PCT/CA2021/050815

§ 371 Date: Dec. 15, 2023

(51) Int. Cl. C07K 1/113; C08F 220/60; C08J 3/075

(52) U.S. Cl. CPC C07K 1/1136; C08F 220/603; C08J 3/075; C08J 2333/26

(57) ABSTRACT

Disclosed herein are hydrogels useful for restoring a biological activity of a denatured polypeptide, solubizing one or more protein aggregates, and/or stabilizing a polypeptide, and methods of using the same, wherein the hydrogel comprises a cross-linked polymer prepared by polymerization of a cross-linker and a first monomer of formula I.

### FIELD OF INVENTION

The present invention relates to restoring, solubilizing and stabilizing polypeptides: more specifically, restoring, solubilizing and stabilizing polypeptides with hydrogels.

### BACKGROUND OF THE INVENTION

Dewatering of biochemical processes is an important step for several industrial applications, such as for drying of gases, for the petrochemical industry, for dehydration of solvents, and in pharmaceutical industries for purification and storage of drugs and proteins. Removal of water in chemical processes is typically achieved by distillation, by membrane reactors, or by molecular sieves.¹ However, application of these methods to bioprocesses is generally not feasible due to the harsh drying conditions and most techniques cannot be applied for the purification and storage of biomolecules based pharmaceutics.³³ Protein-based pharmaceuticals are one example of such bioprocesses, where freeze drying of proteins after their synthesis is the most commonly used method to prolong the shelf life and stability of such therapeutics.³⁴ Freeze drying of proteins can inflict temperature and pressure stresses on the native structure of proteins and can cause their aggregation and loss of biological activities. Furthermore, reconstitution and storage of proteins into liquid forms promotes their aggregation over time, which may reduce the product quality, efficacy and marketability. Current strategies to mitigate the protein aggregation after their reconstitution are to optimize solution conditions (pH, excipient concentrations, and temperature) for each protein to minimize the issues of aggregation and precipitation.³⁴ Such specialized processes are generally cost and time inefficient.

Methods of refolding denatured proteins current involve the use of natural or synthetic chaperones. Synthetic chaperones have been previously used to aid the refolding of thermally or chemically denatured proteins into biologically active forms.¹⁻²² Natural chaperones are biologically active and thermodynamically stable proteins that are typically synthesized as polypeptides, which undergo a series of hydrophobic and ionic interactions to achieve native and functional conformations.^{1.2} This native state of proteins can, however, be disrupted in response to chemical and biological stresses, including aging processes, by the presence of chemicals, heat, and denaturants.

Synthetic chaperones known in the art are typically designed to mimic the GroEL/GroES mechanism of protein refolding and may be composed of polymers,^{4,8,11,12,14-16,22} ionic liquids,⁹ nanoparticles,^{3,7,13,15,19-21} and hydrogels,^{5,6,17} which assist in protein refolding, without them being incorporated into the final folded state of proteins. These macromolecules bind with denatured proteins via hydrophobic or ionic interactions, prevent their aggregation in solution, facilitate the refolding of denatured proteins, and release their biologically active form, as a function of external stimuli such as temperature,^{5,16,21,22} pH,^{16,17} or stripping agents (detergent, cycloamylase, β-cyclodextrin).^{6-8,10,14} Poly(N-isopropylmethacrylamide) (PNIPAM)-based mixed shell polymeric micelles, for example, closely simulate GroEL/GroES complex and confine the denatured proteins at higher temperatures, followed by the release of their refolded forms at lower temperatures.¹⁶ In comparison to nanoparticles, thermally responsive hydrogels interact with denatured proteins via weak noncovalent interactions and solely depend on their thermal flexibility to refold the denatured proteins.³ Kisley et al. have also demonstrated that weak noncovalent interactions between proteins and poly-(acrylamide) hydrogels are sufficient to impart thermal stability to the former and confinement of proteins in the porous architecture of hydrogels has minimum effect on protein stability.²³

There is a clear need for materials that can refold and/or extend the storage life of proteins, such as protein therapeutics, and overcome the current challenges in the art.

### SUMMARY OF THE INVENTION

The present invention relates to stimuli-responsive, poly-(vitamin B5 analgous methacrylamide) P(B5AMA) hydrogels in protein refolding, protein stabilization and storage of proteins. Such smart materials include biomimetic and stimuli responsive poly(B5AMA) hydrogels that are extremely hygroscopic, can absorb copious amount of water from biological solutions, and have exceptional ability to impart thermal stability to the proteins in solution.⁷⁻⁹ For example, poly(B5AMA) hydrogels can selectively absorb water from proteins (lysozyme and carbonic anhydrase) and bacterial solutions, while maintaining the biological activities of biomolecules under temperature and pressure stress (autoclaving and freezing temperatures, freeze drying).

According to one aspect of the present invention, there is provided A hydrogel useful for restoring a biological activity of a denatured polypeptide, wherein the hydrogel comprises a cross-linked polymer prepared by polymerization of a cross-linker and a first monomer of formula I:

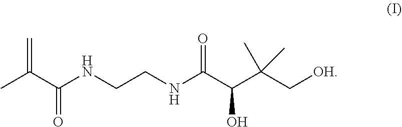 [SMILES: C=C(C)C(=O)NCCNC(=O)[C@H](O)C(C)(C)CO]

According to another aspect of the present invention, there is provided a method for restoring a biological activity of a denatured polypeptide, the method comprising: combining the denatured protein with a hydrogel comprising a cross-linked polymer prepared by polymerization of a cross-linker and a first monomer of formula I:

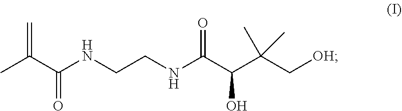 [SMILES: C=C(C)C(=O)NCCNC(=O)[C@H](O)C(C)(C)CO]

and
incubating the denatured protein and the hydrogel.

According to another aspect of the present invention, there is provided a hydrogel useful for solubilizing one or more protein aggregates, wherein the hydrogel comprises a cross-linked polymer prepared by polymerization of a cross-linker and a first monomer of formula I:

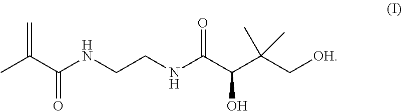 [SMILES: C=C(C)C(=O)NCCNC(=O)[C@H](O)C(C)(C)CO]

According to another aspect of the present invention, there is provided a method for solubilizing one or more protein aggregates, the method comprising: combining the one or more protein aggregates with a hydrogel comprising a cross-linked polymer prepared by polymerization of a cross-linker and a first monomer of formula I:

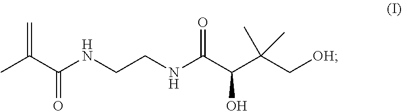 [SMILES: C=C(C)C(=O)NCCNC(=O)[C@H](O)C(C)(C)CO]

and
incubating the one or more protein aggregates and the hydrogel.

According to another aspect of the present invention, there is provided a hydrogel useful for stabilizing a polypeptide, wherein the hydrogel comprises a cross-linked polymer prepared by polymerization of a cross-linker and a first monomer of formula I:

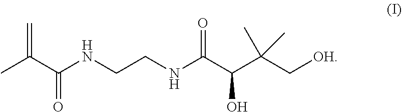 [SMILES: C=C(C)C(=O)NCCNC(=O)[C@H](O)C(C)(C)CO]

According to another aspect of the present invention, there is provided a method of stabilizing a polypeptide, the method comprising: combining the polypeptide with a hydrogel in a solvent, the hydrogel comprising a cross-linked polymer prepared by polymerization of a cross-linker and a first monomer of formula I:

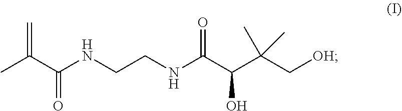 [SMILES: C=C(C)C(=O)NCCNC(=O)[C@H](O)C(C)(C)CO]

and
removing the solvent.

According to another aspect of the present invention, there is provided a method of stabilizing a polypeptide, the method comprising: combining the polypeptide with a hydrogel in a solvent, the hydrogel comprising a cross-linked polymer prepared by polymerization of a cross-linker and a first monomer of formula I:

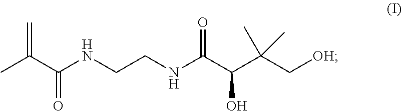 [SMILES: C=C(C)C(=O)NCCNC(=O)[C@H](O)C(C)(C)CO]

and
heating or cooling the solvent.

According to another aspect of the present invention, there is provided a method of stabilizing a polypeptide, the method comprising: coating one or more surfaces of a storage container for the polypeptide with a hydrogel comprising a cross-linked polymer prepared by polymerization of a cross-linker and a first monomer of formula I:

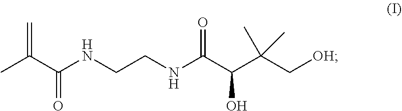 [SMILES: C=C(C)C(=O)NCCNC(=O)[C@H](O)C(C)(C)CO]

and
adding to the storage container the polypeptide.

This summary of the invention does not necessarily describe all features of the invention.

### BRIEF DESCRIPTION OF THE DRAWINGS

These and other features of the invention will become more apparent from the following description in which reference is made to the appended drawings wherein:

FIGS. 1A and 1B are schematics depicting the synthesis of P(B5AMA) hydrogels of different cross-linker densities, using N,N′-Methylene Bisacrylamide as a cross-linker (FIG. 1A), and structural adaptations of P(B5AMA)-10 as a function of temperature (FIG. 1B);

FIGS. 2A and 2B show percent enzymatic activity of lysozyme and carbonic anhydrase in the presence of poly(B5AMA) hydrogel prepared at 5, 10, and 20 mol % cross-linker concentrations (samples are indicated as P(B5AMA)-5, P(B5AMA)-10, and P(B5AMA)-20) (FIG. 2A). Kinetics of lysozyme enzymatic activity (%) in the presence of P(B5AMA)-10, as a function of temperature and time, is shown. The enzymatic activity (%) for proteins is depicted as bar graph, while protein concentrations are plotted as line graph (FIG. 2B);

FIGS. 3A-3C are SEM images showing surface roughness (FIG. 3A), the pore sizes of P(B5AMA) hydrogels as a function of the cross-linker density (FIG. 3B), and a bar graph representing the mean pore sizes of P(B5AMA)-10 and P(B5AMA)-20, using image J software (FIG. 3C). A minimum of 100 pores per hydrogel sample were measured for their sizes;

FIGS. 4A-4C are: a plot of enzymatic activities of lysozyme and carbonic anhydrase upon incubation with P(B5AMA)-10 in native, collapsed, and rehydrated states (FIG. 4A); SEM images visualizing surface roughness of P(B5AMA)-10 hydrogels in both hydrated and collapsed states (FIG. 4B); a plot of pore sizes of P(B5AMA)-10 in both hydrated and collapsed states are measured by image J software and are plotted (FIG. 4C);

FIGS. 5A-5G show enzymatic activities of proteins upon incubation in the presence of dP(B5AMA)-10 and cP(B5AMA)-10 hydrogels are compared with those of native P(B5AMA)-10 hydrogels. The protein enzymatic activities are depicted as bar graphs, while protein concentrations are plotted as line graph. The changes in the pore size and surface texture of P(B5AMA)-10 (FIGS. 5B, 5C), dP(B5AMA)-10 (FIGS. 5D, 5E), and cP(B5AMA)-10 (FIGS. 5F, 5G) are visualized by SEM images;

FIGS. 6A-6D show interactions of biotinylated gold nanoparticles with streptavidin-incubated P(B5AMA)-10 at the surface of hydrogels (FIG. 6A) and in the pores of P(B5AMA)-10 (FIG. 6C); interactions of biotinylated gold nanoparticles with P(B5AMA)-10 in the absence of streptavidin at the surface of hydrogels (FIG. 6B) and in the pores of P(B5AMA)-10 (FIG. 6D);

FIG. 7 is a schematic depicting the synthesis of B5AMA monomer, by ring of opening of D-pantolactone in the presence of 2-aminoethyl methacrylamide (AEMA);

FIG. 8 is a ¹H-NMR spectra of B5AMA in D₂O;

FIGS. 9A and 9B are FTIR spectra of native and heat denatured lysozyme (FIG. 9A) and carbonic anhydrase (FIG. 9B);

FIG. 10 is a Calibration curve of carbonic anhydrase showing the p-nitrophenyl acetate (pNPA) hydrolysis activity of protein, as a function of concentration. The absorbance was recorded at 400 nm after five minutes of incubation;

FIG. 11 is a bar graph depicting denaturation of enzymes in liquid nitrogen, followed by their refolding in the presence of P(B5AMA)-10 hydrogels;

FIG. 12 is a graph depicting a BCA assay of lysozyme prepared at range of concentrations of protein. The absorbance of samples were recorded at 562 nm;

FIG. 13 is a graph depicting a standard calibration curve of TAMRA labelled lysozyme, as a function of concentration of protein. The dye labelled protein solution was excited at 550 nm and emission spectra was recorded at 580 nm;

FIG. 14A depicts lysozyme refolding efficacies of hydrophobic P(B5AMA-co-HxMA)-10 and ionic P(B5AMA-co-MA)-10 are compared with native P(B5AMA)-10. The protein refolding efficacies are depicted as a bar graph, while protein concentrations are plotted as a line graph;

FIG. 14B depicts Coomasie blue staining of hydrogels indicate the entrapment of denatured proteins in matrix of ionic and hydrophobic hydrogels;

FIG. 15 is a graph depicting kinetics of lysozyme enzymatic activities in the presence of P(B5AMA)-10, as a function of time at 37° C. The enzyme activity of protein is depicted as bar graph, while protein concentrations are plotted as line graph;

FIG. 16 depicts enzymatic activity (%) of lysozyme and carbonic anhydrase in the presence of P(B5AMA)-10 hydrogels, as a function of water released from their matrix. Enzymatic activity (%) of proteins are depicted as bar graph, and water released is plotted as line graph;

FIGS. 17A and 17B are SEM images visualizing pore sizes of swollen (FIG. 17A) and collapsed P(B5AMA)-10 (FIG. 17B);

FIG. 18 are FTIR (FIG. 18A) and ¹H-NMR (FIG. 18B) spectra of hydrated and collapsed P(B5AMA)-10 hydrogels;

FIG. 19 is an ATR-FTIR spectra of native and P(B5AMA)-10, acetyl capped cP(B5AMA)-10, and deuterated dP(B5AMA)-10;

FIG. 20 is a ¹H-NMR spectra of 5 kDa HS-PEG-NH₂, biotin-NHS and HS-PEG-biotin conjugates in D₂O;

FIGS. 21A and 21B are schematics depicting the synthesis of hydrogels used in Example 2 (FIG. 21A) and storage stability and dilution of proteins in the presence of hydrogels (FIG. 21B);

FIG. 22 is a graph depicting the cytokine activity of IL-10 and TNF-α solutions after freeze-thawing in the presence or absence of a poly(B5AMA) hydrogel;

FIG. 23 is a graph depicting the cytokine activity of IL-10 and TNF-α solutions after freeze-drying in the presence or absence of a poly(B5AMA) hydrogel;

FIG. 24 is a graph depicting the lysozyme activity of a lysosome solution subjected to heat denaturation in the presence or absence of a poly(B5AMA) hydrogel; and

FIG. 25 is a graph depicting the lysozyme concentration of a lysosome solution subjected to heat denaturation in the presence or absence of a poly(B5AMA) hydrogel.

### DETAILED DESCRIPTION

One or more illustrative embodiments have been described by way of example. Described herein are compositions, methods and uses relating to hydrogels for restoring, solubilizing, and stabilizing polypeptides. It will be appreciated that embodiments and examples are provided for illustrative purposes intended for those skilled in the art, and are not meant to be limiting in any way. All references to embodiments, examples, aspects, formulas, compounds, compositions, solutions, kits and the like is intended to be illustrative and non-limiting.

### Definitions

The term “hydrogel” as used herein will be understood by a person of skill in the art as a 3-dimensional network of polymers. The polymers may be cross-linked and have water dispersed throughout the polymer network. Hydrogels are well-known in the art, for example as described in: Ahmed, E.M. Hydrogel: Preparation, characterization, and applications: A review. Journal of Advanced Research, vol. 6(2), pp. 105-21, which is incorporated herein by reference. The hydrogels described herein are largely comprised of at least B5AMA monomers and are physically cross-linked.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions, to the native sequence, so long as the protein maintains the desired activity. These modifications may be deliberate, as through site-directed mutagenesis, or may be accidental, such as through mutations of hosts which produce the proteins or errors due to PCR amplification. Further, it is noted that the size of polypeptides referenced herein is not particularly limited.

The term “peptide” as used herein refers to a fragment of a polypeptide. Thus, a peptide can include a C-terminal deletion, an N-terminal deletion and/or an internal deletion of the native polypeptide, so long as the entire protein sequence is not present. A peptide will generally include at least about 3-10 contiguous amino acid residues of the full-length molecule, and can include at least about 15-25 contiguous amino acid residues of the full-length molecule, or at least about 20-50 or more contiguous amino acid residues of the full-length molecule, or any integer between 3 amino acids and the number of amino acids in the full-length sequence, provided that the peptide in question retains the ability to elicit the desired biological response.

An “antigen” refers to a molecule, such as a protein, polypeptide, or fragment thereof, containing one or more epitopes (either linear, conformational or both) that will stimulate a host's immune-system to make a humoral and/or cellular antigen-specific response. The term is used interchangeably with the term “immunogen.” Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide which expresses an antigen or antigenic determinant in vivo, such as in DNA immunization applications, is also included in the definition of antigen herein.

An “antibody” intends a molecule that “recognizes,” i.e., specifically binds to an epitope of interest present in an antigen. By “specifically binds” is meant that the antibody interacts with the epitope in a “lock and key” type of interaction to form a complex between the antigen and antibody, as opposed to non-specific binding that might occur between the antibody and, for instance, components in a mixture that includes the test substance with which the antibody is reacted. The term “antibody” as used herein includes antibodies obtained from both polyclonal and monoclonal preparations, as well as, the following: hybrid (chimeric) antibody molecules; F(ab′)₂ and F(ab) fragments; Fv molecules (non-covalent heterodimers; single-chain Fv molecules (sFv); dimeric and trimeric antibody fragment constructs; minibodies; humanized antibody molecules; and, any functional fragments obtained from such molecules, wherein such fragments retain immunological binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)₂, Fv, and other fragments, as well as chimeric and humanized homogeneous antibody populations, that exhibit immunological binding properties of the parent monoclonal antibody molecule.

The term “enzyme” will be understood by a person of skill in the art as a protein or polypeptide that acts as a biological catalyst to accelerate one or more chemical reactions. The term may refer to enzymes that are either folded or denatured, unless otherwise specified.

The term “denature” or “denaturation” will be understood as a protein or polypeptide that has undergone a structural change which results in the loss of its biological properties. The structural change may be a change from a folded or organized structure, such as a quaternary, tertiary or secondary protein structure into a disordered primary structure. Denaturing may be caused by a change in temperature from an ideal temperature, such as a heat or cold shock. This may be understood as “thermal denaturing” as described herein. Thermal denaturing may be any change in temperature that causes a conformational change in the polypeptide such that at least some biological activity is lost. In some cases, the disordered, unfolded primary structure will result in one or more protein aggregates. Such aggregates may be insoluble.

The term “restoring biological activity” as used herein will be understood as refolding a denatured protein such that the biological activity of the enzyme or protein is restored. In some cases, this may be a restoration of catalytic activity. In cases where the denatured protein is an antibody, this may be understood as restoring the antibody's ability to bind its target antigen. By way of example, if the denatured protein is structural protein such as collagen, restoring its biological activity refers to restoring its native folded conformation.

The term “stabilizing a polypeptide” as used herein will be understood as stabilizing a polypeptide such that the polypeptide is less likely to denature or unfold in denaturing conditions, such as thermal denaturing conditions. For example, mixing a hydrogel as described herein with a polypeptide and exposing the mixture to thermal denaturing conditions, such as high heat (>100° C.), will result in less unfolded/degraded polypeptide than if the hydrogel was not present.

The term mol % as used herein will be understood as mole percentage. The mol % will also be understood as the mole fraction of a constituent multiplied by 100%.

### Synthesis of Embodiments of Hydrogels

The synthesis of B5AMA hydrogels has been described previously in the art, for example in International application no. PCT/CA2019/051610, publication no. WO2021092671.

In another embodiment, there is provided herein a method for preparing B5AMA:

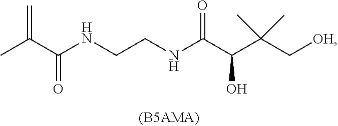 [SMILES: C=C(C)C(=O)NCCNC(=O)[C@H](O)C(C)(C)CO]

said method comprising:
reacting

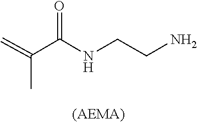

with

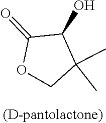

to form B5AMA.

In another embodiment of the above method, the AEMA may be reacted with _{D}-pantolactone in the presence of a weak base. In yet another embodiment of any of the above method or methods, the AEMA may be reacted with _{D}-pantolactone in the presence of the weak base triethylamine (TEA).

In another embodiment, there is provided herein a method for preparing a hygroscopic hydrogel, comprising:
polymerizing one or more monomers in the presence of at least one cross-linker, wherein at least one of the monomers is a compound of formula I:

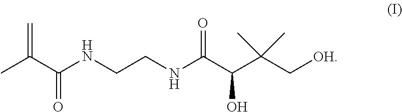 [SMILES: C=C(C)C(=O)NCCNC(=O)[C@H](O)C(C)(C)CO]

In still another embodiment, the one or more monomers may be polymerized by free radical polymerization in the presence of an initiator and a catalyst. The person of skill in the art having regard to the teachings herein will be aware of a wide variety of techniques for polymerizing monomers as described herein, as well as for cross-linking, so as to provide polymers and/or hydrogels suitable for the desired implementation(s) and/or application(s). The skilled person will also understand that one of more functional group(s) of monomers as described herein may be varied or modified to accommodate a desired polymerization technique, if desired. Polymerization techniques, reagents, conditions, and catalysts are known in the art, see for example: Moad, G; Solomon, DH; The Chemistry of Radical Polymerization (Second Edition), Elsevier Science Ltd, 2005, ISBN 9780080442884, herein incorporated by reference in its entirety.

As will be understood, polymerization may typically involve use of an initiator and a catalyst so as to encourage hydrogel formation. The skilled person having regard to the teachings herein will be aware of a wide variety of initiators and catalysts for polymerization, which may be selected based on the monomers and/or conditions being used. By way of example, in an embodiment, an initiator may be selected from KPS, azobisisobutyronitrile (AIBN), VA-044, heat, UV-radiation and others. By way of another example, in an embodiment, a catalyst may be selected from TEMEDA, heat, and others. In an embodiment, the initiator may be KPS, the catalyst may be TEMEDA, or both.

In another embodiment of any of the above method or methods, the polymer generated by monomer polymerization may be cross-linked by the cross-linker as part of the polymerization, or the polymer generated by monomer polymerization may be cross-linked by the cross-linker after monomer polymerization, for example. In certain embodiments, the polymer may be cross-linked by free radical polymerization of the one or more monomers in the presence of one or more cross-linkers, for example.

In still another embodiment of any of the above method or methods, the polymerizing may comprise polymerizing B5AMA monomer:

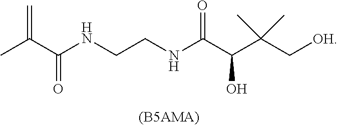 [SMILES: C=C(C)C(=O)NCCNC(=O)[C@H](O)C(C)(C)CO]

In still another embodiment of any of the above method or methods, the polymerizing step may generate a cross-linked polymer which is a homopolymer, or a co-polymer. The compound of formula I may be a monomer of the polymer, and optionally one or more additional monomers may also be used to provide a co-polymer, for example.

In yet another embodiment of any of the above method or methods, the one or more monomers may be polymerized by free radical polymerization in the presence of an initiator and a catalyst. In still another embodiment of any of the above method or methods, the initiator may be KPS, the catalyst may be TEMEDA, or both. In another embodiment of any of the above method or methods, the polymerizing may comprise free radical polymerization.

In another embodiment of any of the above method or methods, the cross-linker may be any one or more of the following cross-linkers:

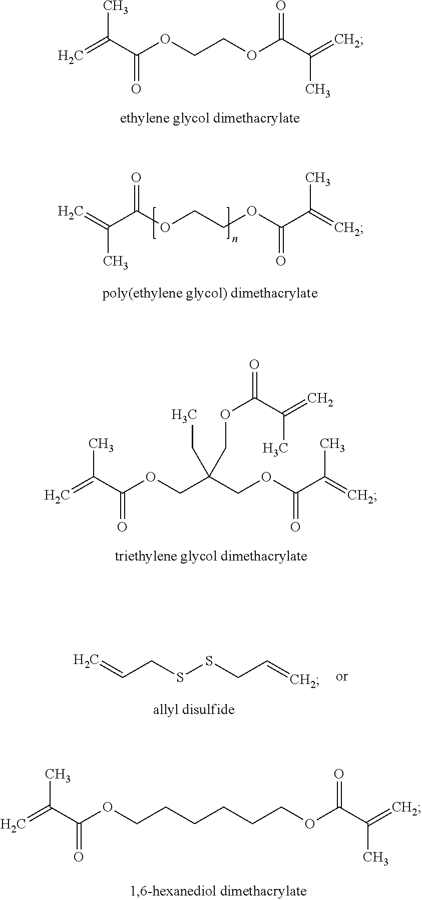

or any combinations thereof.

The skilled person having regard to the teachings herein will be aware of a wide variety of suitable cross-linkers, which may be selected based on the monomers and/or conditions being used and/or desired hydrogel properties. In an embodiment, suitable cross-linkers may include those with two or more “activated” double bonds so as to provide for cross-linking, such as those found in methacrylamide, methacrylate, acrylamide, acrylate, N-vinyl, and other such moieties. Examples of cross-linkers are known in the art, see for example: Moad, G; Solomon, DH; The Chemistry of Radical Polymerization (Second Edition), Elsevier Science Ltd, 2005, ISBN 9780080442884, herein incorporated by reference in its entirety. By way of example, in an embodiment, the cross-linker may be N,N′-methylenebisacrylamide (Bis) cross-linker.

In still another embodiment of any of the above method or methods, the mol % of cross-linker may be a value within a range of about 5 to about 20 mol %, about 5 to about 15 mol %, or about 8 to about 12 mol %, or about 10 mol %.

In another embodiment of any of the above method or methods, at least one of the monomers may be:

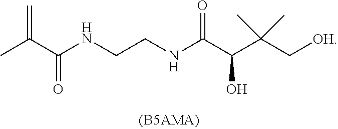 [SMILES: C=C(C)C(=O)NCCNC(=O)[C@H](O)C(C)(C)CO]

In still another embodiment of any of the above method or methods, the polymer may be prepared by polymerization of two or more monomers. The skilled person having regard to the teachings herein will be aware of a wide variety of monomers which may be used for polymerization, which may be selected based on the monomers and/or conditions being used and/or desired properties of resultant hydrogel properties. By way of example, by using a second monomer in addition to the monomer of formula I, it is contemplated that hydrophilic/hydrophobic properties of the hydrogel may be adjusted as desired (see Example 2 below). In certain embodiments, the additional monomers may include those with at least one “activated” double bond so as to allow for polymerization, such as methacrylamide, methacrylate, acrylamide, acrylate, N-vinyl, and other such moieties. By way of example, in an embodiment of any of the above hydrogel or hydrogels, at least one of the monomers may be any one or more of:

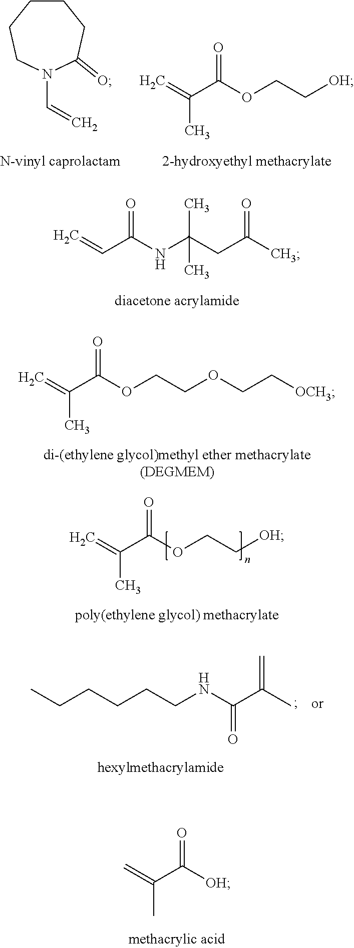

or any combinations thereof.

In yet another embodiment of any of the above method or methods, a molar ratio of the other monomer or monomers to the monomer which is the compound of formula I may be a value within a range of about 0.02 to about 2.

In yet another embodiment of any of the above method or methods, at least one of the monomers may be di-(ethylene glycol)methyl ether methacrylate (DEGMEM). In still another embodiment of any of the above method or methods, the polymerizing step may generate a cross-linked polymer which is a co-polymer, wherein the polymer may be a co-polymer of DEGMEM and B5AMA monomers. In another embodiment of any of the above method or methods, a molar ratio of DEGMEM monomer to B5AMA monomer (DEGMEM/B5AMA) in the polymer may be a value within a range of about 1.1 to 1.5. In still another embodiment of any of the above method or methods, the molar ratio of DEGMEM monomer to B5AMA monomer (DEGMEM/B5AMA) in the polymer may be less than 1.37 or about 1.37.

In another embodiment of any of the above method or methods, the polymer may be prepared by free radical polymerization in the presence of one or more cross-linkers, one or more initiators, and one or more catalysts. In still another embodiment of any of the above method or methods, the cross-linker may be N′,N′-methylene bisacrylamide (Bis), the initiator may be potassium persulfate, the catalyst may be TEMEDA, or any combinations thereof.

The surface of the hydrogel may be modified or functionalized. By way of example, free nucleophilic groups on the surface of the hydrogel, such as amines or hydroxyl groups, may be “capped” via acetic anhydride to provide a non-polar surface. Other functionalization is contemplated, for example adding polar groups such as sugars or adding other functional handles such as streptavidin, biotin or others known in the art.

### Uses and Methods for Restoring Biological Activity of Polypeptides

Disclosed herein are uses of a hydrogel for restoring a biological activity of a denatured polypeptide. Such uses include combining hydrogels as described herein with one or more denatured polypeptides in an aqueous environment. The denatured protein may interact with a part of the hydrogel, for example the surface, and refold to its native confirmation, such that biological activity of the polypeptide is at least partially restored. The denatured polypeptide may be one or more of a protein, an antibody, a cytokine, an enzyme or an antigen. For example, in some embodiments, the denatured polypeptide may be a cytokine such as IL-6, IL-10, and TNF-α, an antibody such as IgG antibodies for ELISA assays, Herceptin, and Cetuximab, a growth factor, a hormone, an enzyme such as lysozyme and carbonic anhydrase, the like, or any combination thereof.

The hydrogel of the embodiments comprises a cross-linked polymer prepared by polymerization of a cross-linker and a first monomer of formula I:

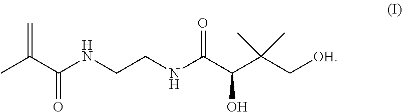 [SMILES: C=C(C)C(=O)NCCNC(=O)[C@H](O)C(C)(C)CO]

In some embodiments, the cross-linked polymer further comprises a second monomer. The second monomer may be:

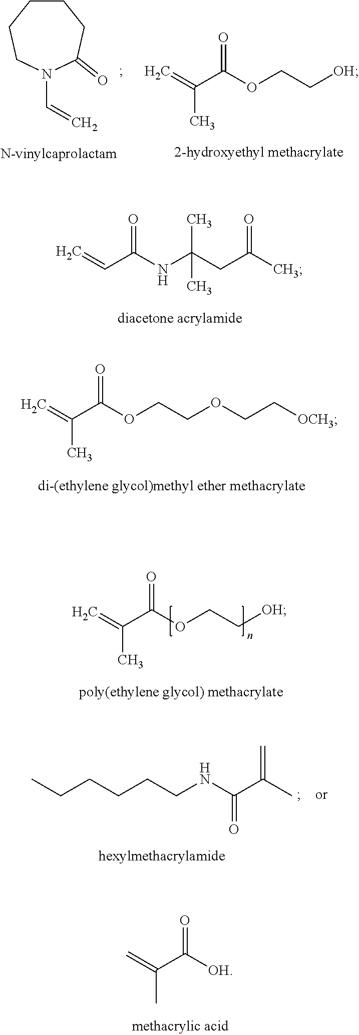

For example, in an embodiment, the second monomer is:

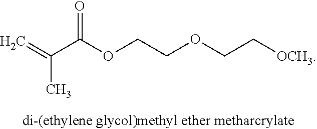

The polymers of the hydrogel are cross-linked with a suitable cross-linker. For example, the cross-linker is N,N′-methylenebisacrylamide (Bis). In some cases, about 0.5 mol % to about 25 mol %, or any value therebetween (optionally rounded to the nearest 0.1), or any subrange spanning between any two of these values, such as 5 mol % to 20 mol % of cross-linker may be used. For example, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.8, 19, 19.5, 20, 20.5, 21, 21.5, 22, 22.5, 23, 23.5, 24, 24.5, 25 mol % or others may be used. In an embodiment, the concentration is about 10 mol % of cross-linker.

Embodiments of the hydrogel are formed via polymerization. Polymerization may be free radical polymerization in the presence of an initiator and a catalyst. For example, the initiator is potassium persulfate (KPS), and the catalyst is N,N,N′,N′-tetramethylethylenediamine (TEMEDA).

The denatured polypeptide may be denatured in denaturing conditions. In some embodiments, the polypeptide is denatured via thermal conditions, such as heating or cooling. Heating may refer to heating to about 110° C. or greater, while cooling may refer to cooling to about −80° C. or lower.

In some cases, heating comprises about 0° C.-150° C. or any value therebetween (optionally rounded to the nearest 0.1), or any subrange spanning between any two of these values, such as 30-110° C. For example, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150° C. or others are considered.

In some cases, cooling comprises about 0° C. to −150° C. or any value therebetween (optionally rounded to the nearest 0.1), or any subrange spanning between any two of these values, such as −10° C. to −80° C. For example, −0.5, −1, −1.5, −2, −2.5, −3, −3.5, −4, −4.5, −5, −5.5, −6, −6.5, −7, −7.5, −8, −8.5, −9, −9.5, −10, −10.5, −11, −11.5, −12, −12.5, −13, −13.5, −14, −14.5, −15, −15.5, −16, −16.5, −17, −17.5, −18, −18.5, −19, −19.5, −20, −20.5, −21, −21.5, −22, −22.5, −23, −23.5, −24, −24.5, −25, −25.5, −26, −26.5, −27, −27.5, −28, −28.5, −29, −29.5, −30, −30.5, −31, −31.5, −32, −32.5, −33, −33.5, −34, −34.5, −35, −35.5, −36, −36.5, −37, −37.5, −38, −38.5, −39, −39.5, −40, −40.5, −41, −41.5, −42, −42.5, −43, −43.5, −44, −44.5, −45, −45.5, −46, −46.5, −47, −47.5, −48, −48.5, −49, −49.5, −50, −51, −52, −53, −54, −55, −56, −57, −58, −59, −60, −61, −62, −63, −64, −65, −66, −67, −68, −69, −70, −71, −72, −73, −74, −75, −76, −77, −78, −79, −80, −81, −82, −83, −84, −85, −86, −87, −88, −89, −90, −91, −92, −93, −94, −95, −96, −97, −98, −99, −100, −101, −102, −103, −104, −105, −106, −107, −108, −109, −110, −111, −112, −113, −114, −115, −116, −117, −118, −119, −120, −121, −122, −123, −124, −125, −126, −127, −128, −129, −130, −131, −132, −133, −134, −135, −136, −137, −138, −139, −140, −141, −142, −143, −144, −145, −146, −147, −148, −149, −150° C. or others are considered.

The hydrogel and the denatured polypeptide are incubated together in solution over a suitable amount of time. In certain embodiments, the suitable amount of time is up to about 24 hours or greater. In an embodiment, the time is 3 hours. In some cases, about 5 minutes to 24 hours or any value therebetween (optionally rounded to the nearest 0.1), or any subrange spanning between any two of these values, such as 1 hour to 3 hours. For example, 0.1, 0.11, 0.12, 0.13, 0.14, 0.15, 0.16, 0.17, 0.18, 0.19, 0.2, 0.21, 0.22, 0.23, 0.24, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.8, 19, 19.5, 20, 20.5, 21, 21.5, 22, 22.5, 23, 23.5, 24 h, or others are considered.

The hydrogel and the denatured polypeptide are incubated together in solution at a suitable temperature. The temperature may be set at the temperature at which the hydrogel extrudes water. In certain embodiments, the temperature is about 4° C. to about 50° C. In an embodiment, the temperature is about 37° C. In some cases, about 1° C. to about 60° C. or any value therebetween (optionally rounded to the nearest 0.1), or any subrange spanning between any two of these values. For example, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50° C. or others are considered.

The hydrogel used in the embodiments has a suitable pore size. For example, the hydrogel may have a pore size of about 4 um to about 10 μm. In an embodiment, the hydrogel has a pore size of about 8 μm. In some cases, about 0.01 μm to about 10 μm or any value therebetween (optionally rounded to the nearest 0.1), or any subrange spanning between any two of these values. For example, 0.01, 0.015, 0.02, 0.025, 0.03, 0.036, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, 0.2, 0.21, 0.22, 0.23, 0.24, 0.25, 0.3, 0.35, 0.4, 0.45, 0.5, 0.55, 0.6, 0.65, 0.7, 0.75, 0.8, 0.85, 0.9, 0.95, 1, 1.01, 1.015, 1.02, 1.025, 1.03, 1.035, 1.04, 1.045, 1.05, 1.055, 1.06, 1.065, 1.07, 1.075, 1.08, 1.085, 1.09, 1.095, 1.1, 1.11, 1.12, 1.13, 1.14, 1.15, 1.16, 1.17, 1.18, 1.19, 1.2, 1.21, 1.22, 1.23, 1.24, 1.25, 1.3, 1.35, 1.4, 1.45, 1.5, 1.55, 1.6, 1.65, 1.7, 1.75, 1.8, 1.85, 1.9, 1.95, 2, 2.1, 2.11, 2.12, 2.13, 2.14, 2.15, 2.16, 2.17, 2.18, 2.19, 2.2, 2.21, 2.22, 2.23, 2.24, 2.25, 2.3, 2.35, 2.4, 2.45, 2.5, 2.55, 2.6, 2.65, 2.7, 2.75, 2.8, 2.85, 2.9, 2.95, 3, 3.1, 3.11, 3.12, 3.13, 3.14, 3.15, 3.16, 3.17, 3.18, 3.19, 3.2, 3.21, 3.22, 3.23, 3.24, 3.25, 3.3, 3.35, 3.4, 3.45, 3.5, 3.55, 3.6, 3.65, 3.7, 3.75, 3.8, 3.85, 3.9, 3.95, 4, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.05, 8.1, 8.11, 8.12, 8.13, 8.14, 8.15, 8.16, 8.17, 8.18, 8.19, 8.2, 8.21, 8.22, 8.23, 8.24, 8.25, 8.3, 8.35, 8.4, 8.45, 8.5, 8.55, 8.6, 8.65, 8.7, 8.75, 8.8, 8.85, 8.9, 8.95, 9, 9.1, 9.2, 9.3, 9.4, 9.5, 9.6, 9.7, 9.8, 9.9, 10 μm, or others are considered.

The denatured polypeptide may be at a suitable concentration when combined with the hydrogel. For example, the concentration of polypeptide is up to about 2 mg per m² of hydrogel. In an embodiment, the concentration is up to about 1.3 mg per m² of hydrogel. In some cases, about 0.01 mg to about 1.3 mg or any value therebetween (optionally rounded to the nearest 0.1), or any subrange spanning between any two of these values. For example, 0.01, 0.015, 0.02, 0.025, 0.03, 0.036, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, 0.2, 0.21, 0.22, 0.23, 0.24, 0.25, 0.3, 0.35, 0.4, 0.45, 0.5, 0.55, 0.6, 0.65, 0.7, 0.75, 0.8, 0.85, 0.9, 0.95, 1, 1.01, 1.015, 1.02, 1.025, 1.03, 1.035, 1.04, 1.045, 1.05, 1.055, 1.06, 1.065, 1.07, 1.075, 1.08, 1.085, 1.09, 1.095, 1, 1.11, 1.115, 1.12, 1.125, 1.13, 1.135, 1.14, 1.145, 1.15, 1.155, 1.16, 1.165, 1.17, 1.175, 1.18, 1.185, 1.19, 1.195, 1.2, 1.21, 1.215, 1.22, 1.225, 1.23, 1.235, 1.24, 1.245, 1.25, 1.255, 1.26, 1.265, 1.27, 1.275, 1.28, 1.285, 1.29, 1.295, 1.3 mg, or others are considered.

Disclosed herein are methods for restoring a biological activity of a denatured polypeptide, the method comprising combining the denatured protein with a hydrogel comprising a cross-linked polymer prepared by polymerization of a cross-linker and a first monomer of formula I:

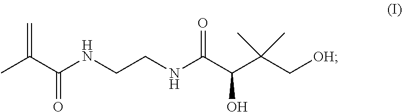 [SMILES: C=C(C)C(=O)NCCNC(=O)[C@H](O)C(C)(C)CO]

and incubating the denatured protein and the hydrogel.

In some embodiments of the method, incubating is at a temperature of about 4° C. to about 50° C. In an embodiment, the temperature is about 37° C. In some cases, about 1° C. to about 60° C. or any value therebetween (optionally rounded to the nearest 0.1), or any subrange spanning between any two of these values. For example, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50° C. or others are considered.

The hydrogel and the denatured polypeptide are incubated together in solution over a suitable amount of time. In certain embodiments, the suitable amount of time is up to about 24 hours or greater. In an embodiment, the time is 3 hours. In some cases, about 5 minutes to 24 hours or any value therebetween (optionally rounded to the nearest 0.1), or any subrange spanning between any two of these values, such as 1 hour to 3 hours. For example, 0.1, 0.11, 0.12, 0.13, 0.14, 0.15, 0.16, 0.17, 0.18, 0.19, 0.2, 0.21, 0.22, 0.23, 0.24, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.8, 19, 19.5, 20, 20.5, 21, 21.5, 22, 22.5, 23, 23.5, 24 h, or others are considered.

### Uses and Methods for Solubilizing Protein Aggregates

Disclosed herein are methods and uses of a hydrogel for solubilizing one or more protein aggregates. Such uses include combining hydrogels as described herein with one or more protein aggregates. The aggregates may comprise one or more denatured proteins, such that the proteins have an unstable conformation and have crashed out of an aqueous solution. Solubilizing the aggregates may be achieved by refolding the one or more proteins within the aggregate such that they become soluble. In some cases, this is caused by exposing polar or hydrophilic residues to the surface of the protein and “burying” the hydrophobic residues away from the aqueous solvent. The protein aggregate may comprise one or more of a protein, an antibody, a cytokine, an enzyme or an antigen. For example, in some embodiments, the denatured polypeptide may be a cytokine such as IL-6, IL-10, and TNF-α, an antibody such as IgG antibodies for ELISA assays, Herceptin, and Cetuximab, a growth factor, a hormone, an enzyme such as lysozyme and carbonic anhydrase, the like, or any combination thereof.

The hydrogel of the embodiments comprises a cross-linked polymer prepared by polymerization of a cross-linker and a first monomer of formula I:

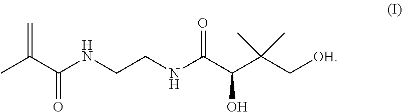 [SMILES: C=C(C)C(=O)NCCNC(=O)[C@H](O)C(C)(C)CO]

In some embodiments, the cross-linked polymer further comprises a second monomer. The second monomer may be:

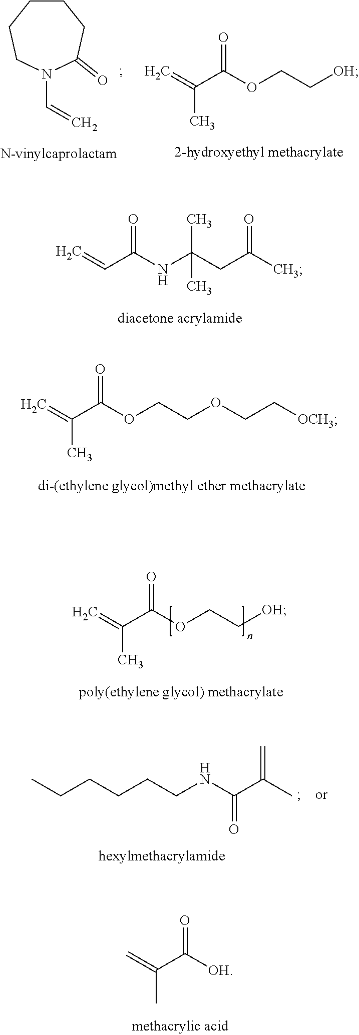

For example, in an embodiment, the second monomer is:

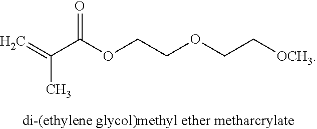

The polymers of the hydrogel are cross-linked with a suitable cross-linker. For example, the cross-linker is N,N′-methylenebisacrylamide (Bis). In some cases, about 0.5 mol % to about 25 mol %, or any value therebetween (optionally rounded to the nearest 0.1), or any subrange spanning between any two of these values, such as 5 mol % to 20 mol % of cross-linker may be used. For example, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.8, 19, 19.5, 20, 20.5, 21, 21.5, 22, 22.5, 23, 23.5, 24, 24.5, 25 mol % or others may be used. In an embodiment, the concentration is about 10 mol % of cross-linker.

Embodiments of the hydrogel are formed via polymerization. Polymerization may be free radical polymerization in the presence of an initiator and a catalyst. For example, the initiator is potassium persulfate (KPS), and the catalyst is N,N,N′,N′-tetramethylethylenediamine (TEMEDA).

The one or more protein aggregates may have formed in denaturing conditions. In some embodiments, the one or more protein aggregates is formed via thermal conditions, such as heating or cooling. Heating may refer to heating to about 110° C. or greater, while cooling may refer to cooling to about −80° C. or lower.

In some cases, heating comprises about 0° C.-150° C. or any value therebetween (optionally rounded to the nearest 0.1), or any subrange spanning between any two of these values, such as 30-110° C. For example, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150° C. or others are considered.

In some cases, cooling comprises about 0° C. to −150° C. or any value therebetween (optionally rounded to the nearest 0.1), or any subrange spanning between any two of these values, such as −10° C. to −80° C. For example, −0.5, −1, −1.5, −2, −2.5, −3, −3.5, −4, −4.5, −5, −5.5, −6, −6.5, −7, −7.5, −8, −8.5, −9, −9.5, −10, −10.5, −11, −11.5, −12, −12.5, −13, −13.5, −14, −14.5, −15, −15.5, −16, −16.5, −17, −17.5, −18, −18.5, −19, −19.5, −20, −20.5, −21, −21.5, −22, −22.5, −23, −23.5, −24, −24.5, −25, −25.5, −26, −26.5, −27, −27.5, −28, −28.5, −29, −29.5, −30, −30.5, −31, −31.5, −32, −32.5, −33, −33.5, −34, −34.5, −35, −35.5, −36, −36.5, −37, −37.5, −38, −38.5, −39, −39.5, −40, −40.5, −41, −41.5, −42, −42.5, −43, −43.5, −44, −44.5, −45, −45.5, −46, −46.5, −47, −47.5, −48, −48.5, −49, −49.5, −50, −51, −52, −53, −54, −55, −56, −57, −58, −59, −60, −61, −62, −63, −64, −65, −66, −67, −68, −69, −70, −71, −72, −73, −74, −75, −76, −77, −78, −79, −80, −81, −82, −83, −84, −85, −86, −87, −88, −89, −90, −91, −92, −93, −94, −95, −96, −97, −98, −99, −100, −101, −102, −103, −104, −105, −106, −107, −108, −109, −110, −111, −112, −113, −114, −115, −116, −117, −118, −119, −120, −121, −122, −123, −124, −125, −126, −127, −128, −129, −130, −131, −132, −133, −134, −135, −136, −137, −138, −139, −140, −141, −142, −143, −144, −145, −146, −147, −148, −149, −150° C. or others are considered.

The hydrogel and the one or more protein aggregates are incubated together in solution over a suitable amount of time. In certain embodiments, the suitable amount of time is up to about 24 hours or greater. In an embodiment, the time is 3 hours. In some cases, about 5 minutes to 24 hours or any value therebetween (optionally rounded to the nearest 0.1), or any subrange spanning between any two of these values, such as 1 hour to 3 hours. For example, 0.1, 0.11, 0.12, 0.13, 0.14, 0.15, 0.16, 0.17, 0.18, 0.19, 0.2, 0.21, 0.22, 0.23, 0.24, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.8, 19, 19.5, 20, 20.5, 21, 21.5, 22, 22.5, 23, 23.5, 24 h, or others are considered.

The hydrogel and the one or more protein aggregates are incubated together in solution at a suitable temperature. The temperature may be set at the temperature at which the hydrogel extrudes water. In certain embodiments, the temperature is about 4° C. to about 50° C. In an embodiment, the temperature is about 37° C. In some cases, about 1° C. to about 60° C. or any value therebetween (optionally rounded to the nearest 0.1), or any subrange spanning between any two of these values. For example, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50° C. or others are considered.

The hydrogel used in the embodiments has a suitable pore size. For example, the hydrogel may have a pore size of about 4 μm to about 10 μm. In an embodiment, the hydrogel has a pore size of about 8 μm. In some cases, about 0.01 μm to about 10 um or any value therebetween (optionally rounded to the nearest 0.1), or any subrange spanning between any two of these values. For example, 0.01, 0.015, 0.02, 0.025, 0.03, 0.036, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, 0.2, 0.21, 0.22, 0.23, 0.24, 0.25, 0.3, 0.35, 0.4, 0.45, 0.5, 0.55, 0.6, 0.65, 0.7, 0.75, 0.8, 0.85, 0.9, 0.95, 1, 1.01, 1.015, 1.02, 1.025, 1.03, 1.035, 1.04, 1.045, 1.05, 1.055, 1.06, 1.065, 1.07, 1.075, 1.08, 1.085, 1.09, 1.095, 1.1, 1.11, 1.12, 1.13, 1.14, 1.15, 1.16, 1.17, 1.18, 1.19, 1.2, 1.21, 1.22, 1.23, 1.24, 1.25, 1.3, 1.35, 1.4, 1.45, 1.5, 1.55, 1.6, 1.65, 1.7, 1.75, 1.8, 1.85, 1.9, 1.95, 2, 2.1, 2.11, 2.12, 2. 13, 2.14, 2.15, 2.16, 2.17, 2.18, 2.19, 2.2, 2.21, 2.22, 2.23, 2.24, 2.25, 2.3, 2.35, 2.4, 2.45, 2.5, 2.55, 2.6, 2.65, 2.7, 2.75, 2.8, 2.85, 2.9, 2.95, 3, 3.1, 3.11, 3.12, 3.13, 3.14, 3.15, 3.16, 3.17, 3.18, 3.19, 3.2, 3.21, 3.22, 3.23, 3.24, 3.25, 3.3, 3.35, 3.4, 3.45, 3.5, 3.55, 3.6, 3.65, 3.7, 3.75, 3.8, 3.85, 3.9, 3.95, 4, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.05, 8.1, 8.11, 8.12, 8.13, 8.14, 8.15, 8.16, 8.17, 8.18, 8.19, 8.2, 8.21, 8.22, 8.23, 8.24, 8.25, 8.3, 8.35, 8.4, 8.45, 8.5, 8.55, 8.6, 8.65, 8.7, 8.75, 8.8, 8.85, 8.9, 8.95, 9, 9.1, 9.2, 9.3, 9.4, 9.5, 9.6, 9.7, 9.8, 9.9, 10 μm, or others are considered.

The denatured polypeptide may be at a suitable concentration when combined with the hydrogel. For example, the concentration of polypeptide is up to about 2 mg per m² of hydrogel. In an embodiment, the concentration is up to about 1.3 mg per m² of hydrogel. In some cases, about 0.01 mg to about 1.3 mg or any value therebetween (optionally rounded to the nearest 0.1), or any subrange spanning between any two of these values. For example, 0.01, 0.015, 0.02, 0.025, 0.03, 0.036, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, 0.2, 0.21, 0.22, 0.23, 0.24, 0.25, 0.3, 0.35, 0.4, 0.45, 0.5, 0.55, 0.6, 0.65, 0.7, 0.75, 0.8, 0.85, 0.9, 0.95, 1, 1.01, 1.015, 1.02, 1.025, 1.03, 1.035, 1.04, 1.045, 1.05, 1.055, 1.06, 1.065, 1.07, 1.075, 1.08, 1.085, 1.09, 1.095, 1, 1.11, 1.115, 1.12, 1.125, 1.13, 1.135, 1.14, 1.145, 1.15, 1.155, 1.16, 1.165, 1.17, 1.175, 1.18, 1.185, 1.19, 1.195, 1.2, 1.21, 1.215, 1.22, 1.225, 1.23, 1.235, 1.24, 1.245, 1.25, 1.255, 1.26, 1.265, 1.27, 1.275, 1.28, 1.285, 1.29, 1.295, 1.3 mg, or others are considered.

Disclosed herein are methods for solubilizing one or more protein aggregates, the method comprising combining one or more protein aggregates with a hydrogel comprising a cross-linked polymer prepared by polymerization of a cross-linker and a first monomer of formula I:

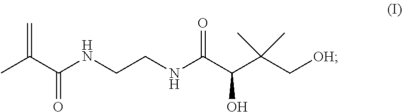 [SMILES: C=C(C)C(=O)NCCNC(=O)[C@H](O)C(C)(C)CO]

and incubating the one or more protein aggregates and the hydrogel.

In some embodiments of the method, incubating is at a temperature of about 4° C. to about 50° C. In an embodiment, the temperature is about 37° C. In some cases, about 1° C. to about 60° C. or any value there between (optionally rounded to the nearest 0.1), or any subrange spanning between any two of these values. For example, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50° C. or others are considered.

The hydrogel and the one or more protein aggregates are incubated together in solution over a suitable amount of time. In certain embodiments, the suitable amount of time is up to about 24 hours or greater. In an embodiment, the time is 3 hours. In some cases, about 5 minutes to 24 hours or any value there between (optionally rounded to the nearest 0.1), or any subrange spanning between any two of these values, such as 1 hour to 3 hours. For example, 0.1, 0.11, 0.12, 0.13, 0.14, 0.15, 0.16, 0.17, 0.18, 0.19, 0.2, 0.21, 0.22, 0.23, 0.24, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.8, 19, 19.5, 20, 20.5, 21, 21.5, 22, 22.5, 23, 23.5, 24 h, or others are considered.

### Uses and Methods for Stabilizing a Polypeptide

Described herein are uses and methods of a hydrogel for stabilizing a polypeptide. In some cases, a folded, native and/or soluble protein is mixed with a hydrogel as described herein. The hydrogel may act to stabilize or decrease the amount of unfolding and/or denaturing of the polypeptide when exposed to denaturing conditions, such as temperature, pressure or extended periods of time. In some embodiments, the hydrogel is used to extend a shelf-life of a protein. In other embodiments, the hydrogel is used to dilute the protein and prevent formation of aggregates. The polypeptide may be one or more of a protein, an antibody, a cytokine, an enzyme or an antigen. For example, in some embodiments, the denatured polypeptide may be a cytokine such as IL-6, IL-10, and TNF-α, an antibody such as IgG antibodies for ELISA assays, Herceptin, and Cetuximab, a growth factor, a hormone, an enzyme such as lysozyme and carbonic anhydrase, the like, or any combination thereof.

The hydrogel of the embodiments comprises a cross-linked polymer prepared by polymerization of a cross-linker and a first monomer of formula I:

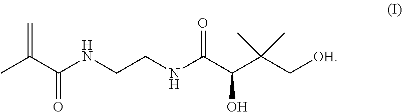 [SMILES: C=C(C)C(=O)NCCNC(=O)[C@H](O)C(C)(C)CO]

In some embodiments, the cross-linked polymer further comprises a second monomer. The second monomer may be:

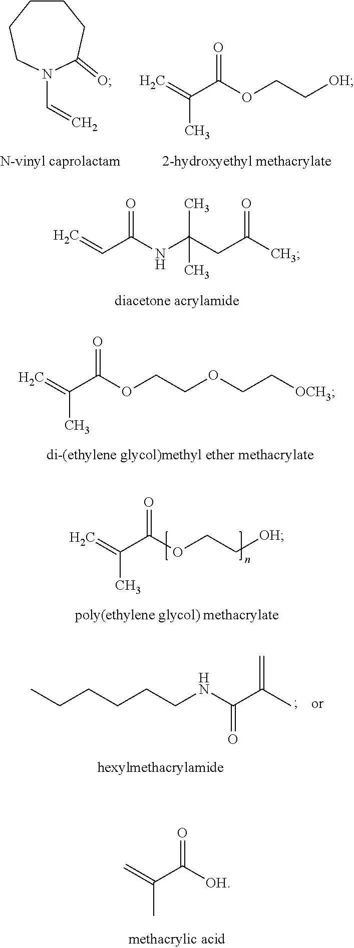

For example. in an embodiment. the second monomer is:

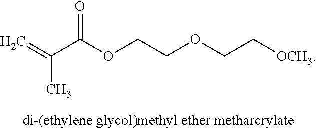

The polymers of the hydrogel are cross-linked with a suitable cross-linker. For example, the cross-linker is N,N′-methylenebisacrylamide (Bis). In some cases, about 0.5 mol % to about 25 mol %, or any value therebetween (optionally rounded to the nearest 0.1), or any subrange spanning between any two of these values, such as 5 mol % to 20 mol % of cross-linker may be used. For example, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.8, 19, 19.5, 20, 20.5, 21, 21.5, 22, 22.5, 23, 23.5, 24, 24.5, 25 mol % or others may be used. In an embodiment, the concentration is about 10 mol % of cross-linker.

Embodiments of the hydrogel are formed via polymerization. Polymerization may be free radical polymerization in the presence of an initiator and a catalyst. For example, the initiator is potassium persulfate (KPS), and the catalyst is N,N,N′,N′-tetramethylethylenediamine (TEMEDA).

The polypeptide may be at a suitable concentration when combined with the hydrogel. For example, the concentration of polypeptide is up to about 2 mg per m² of hydrogel. In an embodiment, the concentration is up to about 1.3 mg per m² of hydrogel. In some cases, about 0.01 mg to about 1.3 mg or any value therebetween (optionally rounded to the nearest 0.1), or any subrange spanning between any two of these values. For example, 0.01, 0.015, 0.02, 0.025, 0.03, 0.036, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, 0.2, 0.21, 0.22, 0.23, 0.24, 0.25, 0.3, 0.35, 0.4, 0.45, 0.5, 0.55, 0.6, 0.65, 0.7, 0.75, 0.8, 0.85, 0.9, 0.95, 1, 1.01, 1.015, 1.02, 1.025, 1.03, 1.035, 1.04, 1.045, 1.05, 1.055, 1.06, 1.065, 1.07, 1.075, 1.08, 1.085, 1.09, 1.095, 1, 1.11, 1.115, 1.12, 1.125, 1.13, 1.135, 1.14, 1.145, 1.15, 1.155, 1.16, 1.165, 1.17, 1.175, 1.18, 1.185, 1.19, 1.195, 1.2, 1.21, 1.215, 1.22, 1.225, 1.23, 1.235, 1.24, 1.245, 1.25, 1.255, 1.26, 1.265, 1.27, 1.275, 1.28, 1.285, 1.29, 1.295, 1.3 mg, or others are considered.

In some embodiments of the uses and methods described herein, the hydrogel and the polypeptide are dried after combining/mixing. For example, the hydrogel and polypeptide may be freeze-dried after combining/mixing. In a further embodiment, the hydrogel and polypeptide may be dried after a suitable incubation period. . . . By way of a further example, the hydrogel and a protein aggregate may be combined in water, incubated for a suitable period, such as 3 hours, at a suitable temperature, such as 37° C., prior to drying. In such embodiments the protein aggregate is given time to associate to the surface of the hydrogel and untangle prior to drying by a suitable method, such as freeze drying. After the protein aggregate and hydrogel are dried, they may be reconstituted in aqueous solvent.

The polypeptide may be heated with the hydrogel, either dried or in aqueous solvent. In some cases, heating comprises about 0° C.-150° C. or any value therebetween (optionally rounded to the nearest 0.1), or any subrange spanning between any two of these values, such as 30-110° C. For example, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150° C. or others are considered.

Methods of stabilizing a polypeptide are disclosed herein. Embodiments of the method comprise combining the polypeptide with a hydrogel in a solvent, the hydrogel comprising a cross-linked polymer prepared by polymerization of a cross-linker and a first monomer of formula I:

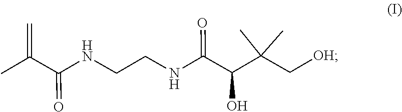 [SMILES: C=C(C)C(=O)NCCNC(=O)[C@H](O)C(C)(C)CO]

and removing the solvent. In some embodiments, the solvent is removed via freeze-drying.

Further embodiments of the methods for stabilizing a polypeptide comprise, combining the polypeptide with a hydrogel in a solvent, the hydrogel comprising a cross-linked polymer prepared by polymerization of a cross-linker and a first monomer of formula I:

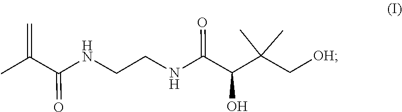 [SMILES: C=C(C)C(=O)NCCNC(=O)[C@H](O)C(C)(C)CO]

and heating or cooling the solvent.

Heating may comprise heating to about 110° C. In some cases, heating comprises about 0° C.-150° C. or any value therebetween (optionally rounded to the nearest 0.1), or any subrange spanning between any two of these values, such as 30-110° C. For example, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150° C. or others are considered.

Cooling may comprise cooling to about −80° C. In some cases, cooling comprises about 0° C. to-150° C. or any value therebetween (optionally rounded to the nearest 0.1), or any subrange spanning between any two of these values, such as −10° C. to −80° C. For example, −0.5, −1, −1.5, −2, −2.5, −3, −3.5, −4, −4.5, −5, −5.5, −6, −6.5, −7, −7.5, −8, −8.5, −9, −9.5, −10, −10.5, −11, −11.5, −12, −12.5, −13, −13.5, −14, −14.5, −15, −15.5, −16, −16.5, −17, −17.5, −18, −18.5, −19, −19.5, −20, −20.5, −21, −21.5, −22, −22.5, −23, −23.5, −24, −24.5, −25, −25.5, −26, −26.5, −27, −27.5, −28, −28.5, −29, −29.5, −30, −30.5, −31, −31.5, −32, −32.5, −33, −33.5, −34, −34.5, −35, −35.5, −36, −36.5, −37, −37.5, −38, −38.5, −39, −39.5, −40, −40.5, −41, −41.5, −42, −42.5, −43, −43.5, −44, −44.5, −45, −45.5, −46, −46.5, −47, −47.5, −48, −48.5, −49, −49.5, −50, −51, −52, −53, −54, −55, −56, −57, −58, −59, −60, −61, −62, −63, −64, −65, −66, −67, −68, −69, −70, −71, −72, −73, −74, −75, −76, −77, −78, −79, −80, −81, −82, −83, −84, −85, −86, −87, −88, −89, −90, −91, −92, −93, −94, −95, −96, −97, −98, −99, −100, −101, −102, −103, −104, −105, −106, −107, −108, −109, −110, −111, −112, −113, −114, −115, −116, −117, −118, −119, −120, −121, −122, −123, −124, −125, −126, −127, −128, −129, −130, −131, −132, −133, −134, −135, −136, −137, −138, −139, −140, −141, −142, −143, −144, −145, −146, −147, −148, −149, −150° C. or others are considered.

The above embodiments may be particularly useful for laboratory-grade polypeptides. As will be appreciated, laboratory-grade polypeptides often undergo multiple freeze-thaw cycles during use thereof, which, as discussed above, may denature the polypeptides over time. Use of the hydrogels of the present disclosure may therefore prevent or reduce the negative effects (e.g. denaturing) caused by the freeze-thaw cycles, thereby preserving the polypeptides.

Further, the above embodiments may also be particularly useful for vaccine storage and distribution. In more detail, certain types of vaccines often have to be stored at extreme temperatures such as −20° C. or lower to prevent the denaturing of the polypeptides contained therein. As will be appreciated, specialized, expensive equipment is required to maintain such temperatures. Use of the hydrogels of the present disclosure may avoid the need for such specialized equipment by preventing or reducing the likelihood of denaturing of the polypeptides of the vaccines at elevated temperatures.

It is also noted that the hydrogels of the present disclosure may be used to preserve a polypeptide at any point in the production process thereof. For example, in some embodiments, the polypeptide may be produced, stored, and shipped and then subsequently mixed with a hydrogel of the present disclosure for the stabilization thereof. Alternatively, in other embodiments, the polypeptide may be added to a hydrogel of the present disclosure immediately after production for immediate stabilization and preservation thereof.

For example, in some embodiments, one or more internal surfaces of a storage container for storing therein a polypeptide may be coated with a hydrogel of the present disclosure. In such embodiments, a polypeptide may be added to a hydrogel of the present disclosure immediately after the production thereof

The coating of the storage containers with the hydrogels of the present disclosure may be performed a number of ways. For example, in embodiments where the storage container is formed of glass, one or more of the internal surfaces of the storage container may be modified to introduce functional groups that are bondable to the hydrogels of the present disclosure. In one non-limiting embodiment, the internal surfaces of glass storage containers may be first modified to introduce primary amine groups thereon and then subsequently modified to introduce alkene groups thereon. The hydrogels of the present disclosure may then be polymerized and crosslinked on the modified surfaces.

The surfaces may be modified using any suitable reagent. As will be appreciated, the regents selected may depend on the composition of the hydrogels to be used. For example, a reagent such as 3-amiopropyl triethoxysilane (APTES) may be used to introduce primary amine groups onto the surfaces of the storage container and a reagent such as methacrylic acid may be used to subsequently introduce alkene groups.

Alternatively, in some embodiments, the hydrogels may be coated onto one or more surfaces of the storage containers without the modifying thereof.

Further, it is noted that the storage containers may be any type of container conventionally used for storing polypeptides. For example, the storage containers may be test tubes, vials, bottles, or the like. The storage containers may be formed of any material used in industry, such as a plastic or glass.

Once the one or more surfaces of the storage container are coated with a hydrogel of the present disclosure, a polypeptide may be added thereto. Coating one or more surfaces of a storage container with a hydrogel may be particularly useful for upscaling the methods of the present disclosure to industrially relevant levels. For example, in some embodiments, the addition of the polypeptide to a hydrogel-coated storage container may be performed as a batch process. For example, the polypeptide may be added to the hydrogel-coated storage container and subsequently processed as previously described herein (e.g. heated, freeze-dried, etc.) in one or more batch reactors.

The present invention will be further illustrated in the following examples.

### EXAMPLES

Example 1: Elucidating the Role of Thermal Flexibility of Hydrogels in Protein Refolding

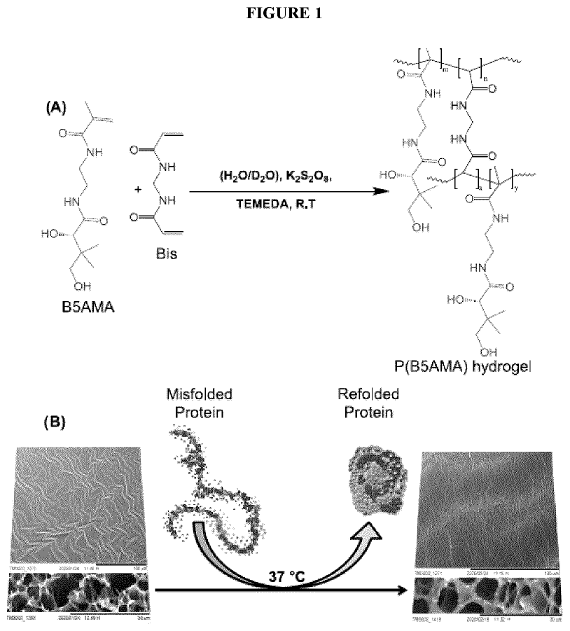

In this study, this temperature-and water-dependent oscillatory behavior of P(B5AMA) hydrogels is utilized to evaluate the physical and structural changes that may participate in protein refolding efficacies of thermoresponsive hydrogels. This Example will demonstrate the physical changes in the structure of hydrogels as a function of temperature and their correlation with protein refolding efficacies. To study the refolding of thermally denatured proteins in the presence of stimuli-responsive hydrogels, P(B5AMA) hydrogels of different net charges, hydrophobicity, and cross-linking densities are synthesized by radical polymerization method (FIGS. 1A, 6, and Table 1) and are evaluated for the restoration of enzymatic activity of thermally denatured enzymes (lysozyme and carbonic anhydrase) as a function of temperature, time, and presence of residual water in the hydrogel architecture. The hydrogels with promising protein refolding efficacies are further evaluated for their interactions with denatured proteins, and the role of the hydrogel matrix in protein refolding capabilities is elucidated.

The hydrogels disclosed herein operate as a function of one or more of: surface chemistry, pore sizes, and temperature-responsive behavior. In comparison to other thermoresponsive systems, which oscillate between hydrophilic and hydrophobic states as a function of temperature,^{5,16,21,22} poly(B5AMA) hydrogels release loosely bound water from their architecture at a higher temperature (such as >35° C.) and may get trapped in an irreversible collapsed form.²⁴ This collapsed state of poly-(B5AMA) hydrogels may be maintained at room temperature for weeks, in the absence of water lost from the hydrogel architecture, and reversible oscillation of hydrogels into the hydrophilic state requires their re-swelling at lower temperatures (such as <35° C.).²⁴

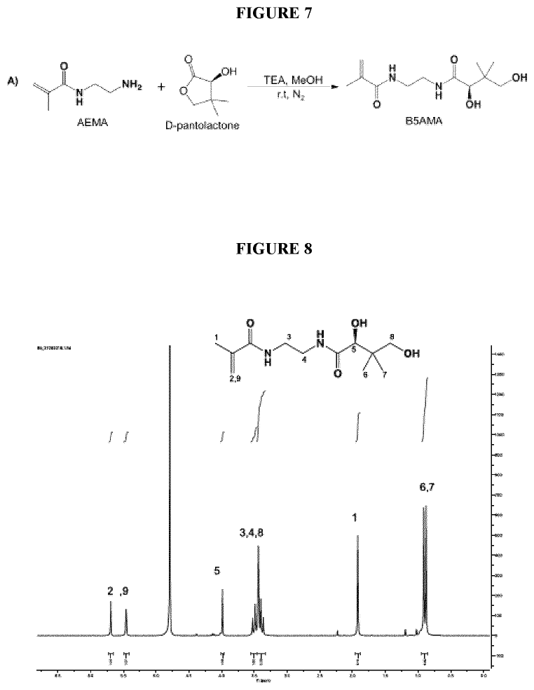

P(B5AMA) hydrogels of different cross-linking densities were prepared by free-radical polymerization of the B5AMA monomer in the presence of different concentrations of N,N′-methylene bisacrylamide as a cross-linker (FIGS. 1A, 7, 8 and Table 1. The synthesis of hydrogels of different cross-linker densities (5, 10, and 20 mol % cross-linkers, indicated as P(B5AMA)-5, P(B5AMA)-10, and P(B5AMA)-20, respectively) was confirmed by vial inversion test, and the percent yield for P(B5AMA)-5, P(B5AMA)-10, and P(B5AMA)-20 was found to be 66, 74, and 85%, respectively.

TABLE 1
Poly(B5AMA) hydrogels were synthesized by free-radical polymerization of B5AMA monomer, in the presence
of different concentrations of N,N′-methylene bisacrylamide (as cross-linker), co-monomers, and
solvent and were surface-modified by acetic anhydride to modulate the hydrogen-bonding interactions.

| | | | cross-linker | |
|---|---|---|---|---|
| | B5AMA | co-monomer (mol %)/hydrogel | concentration | |
| sample name | [M] | modification | (mol %) | solvent |
| P(B5AMA)-5 | 0.78 | | 5 | deionized water |
| P(B5AMA)-10 | 0.78 | | 10 | deionized water |
| P(B5AMA)-20 | 0.78 | | 20 | deionized water |
| P(B5AMA-co-HxMA)-10 | 0.78 | 10 mol % hexylmethacrylamide | 10 | deionized water |
| P(B5AMA-co-MA)-10 | 0.78 | 10 mol % methacrylic acid | 10 | deionized water |
| cP(B5AMA)-10 | 0.78 | acetic anhydride capping of OH groups | 10 | deionized water |
| dP(B5AMA)-10 | 0.78 | | 10 | deuterated water |

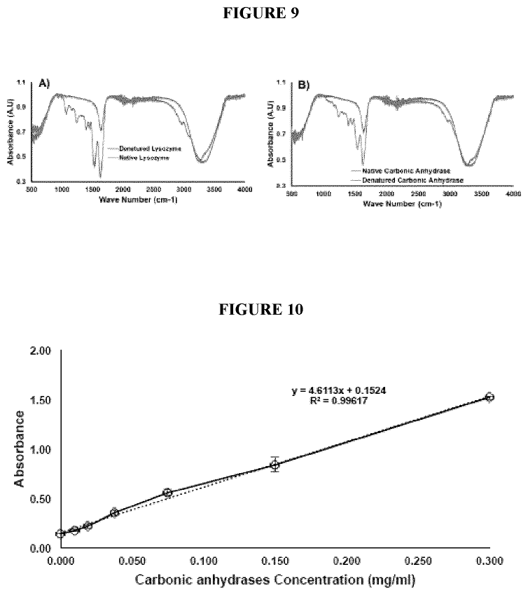

P(B5AMA)-5, P(B5AMA)-10, and P(B5AMA)-20 prepared were tested for restoration of enzymatic activities of heat-denatured proteins, using lysozyme and carbonic anhydrase, as model proteins. Preliminary data indicated that thermal denaturation of lysozymes near autoclave temperatures (110° C.) significantly reduced the activity of enzymes to 4±0.02%. Heat denaturation of protein at an autoclaving temperature resulted in physical changes in protein structures, and aggregation of proteins in an aqueous solution was observed by DLS and ATR-FTIR spectroscopy (FIG. 9 and Table 2). Native enzymes were completely soluble in aqueous buffers, with sizes ranging from 2 to 10 nm in diameter, and showed no distinct peaks in FTIR spectra. The heating of carbonic anhydrase and lysozyme in aqueous buffers, however, resulted in the formation of aggregates with sizes ranging from 150 to 300 nm in diameter, and several distinct peaks were observed in FTIR spectra, indicating the aggregation of proteins due to the appearance of hydrophobic residues in solution. The presence of P(B5AMA) hydrogels during heat denaturation of lysozyme helped stabilize the native structure of proteins, and the enzymatic activity of lysozyme was 66±3.5%, in the presence of P(B5AMA)-10 (Table 3). Moreover, the thermal stability of lysozyme in the presence of P(B5AMA) hydrogels was independent of the cross-linker concentration of hydrogels. These results are consistent with the previous studies, where poly(acrylamide) hydrogels of different cross-linking densities are shown to improve the thermal stability of proteins at high temperatures.²³

TABLE 2
DLS measurement of hydrodynamic diameter
of native and heat denatured proteins.

| | Hydrodynamic | |
|---|---|---|
| Sample | Diameter (nm) | Polydispersity Index |
| Native Lysozyme | 3.8 | 0.08 |
| Denatured Lysozyme | 164.9 | 0.39 |
| Carbonic Anhydrase | 2.13 | 0.33 |
| Denatured Carbonic | 381.7 | 0.55 |
| Anhydrase | | |

TABLE 3
Percent activity of lysozyme after heating at
110° C. in the presence or absence of P(B5AMA)
hydrogels of different cross-linker densities.

| Sample | % Lysozyme activity |
|---|---|
| PBS | 4 ± 0.02 |
| P(B5AMA)-5 | 53.1 ± 2.6 |
| P(B5AMA)-10 | 66.7 ± 3.1 |
| P(B5AMA)-20 | 61.7 ± 2.0 |

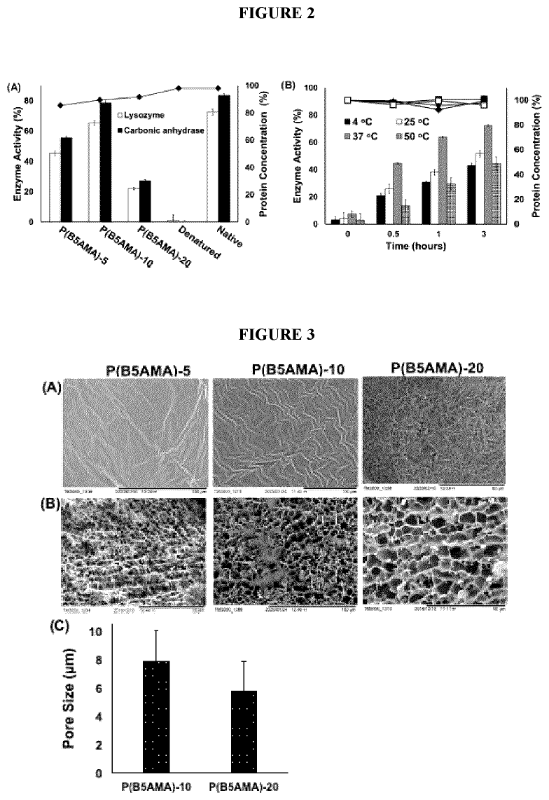

P(B5AMA) hydrogels are an example stimuli-responsive material, where temperature-responsive behavior of hydrogels is dependent on the hydrogen-bonding interactions between water molecules and hydrogel matrix and can be tuned as a function of their cross-linker concentration.²⁴ This cross-linker concentration-dependent hydrogen-bonding capabilities of P(B5AMA) hydrogels have been utilized to release loosely bound water from their polymeric architecture and shown that water release efficacies of P(B5AMA)-10 hydrogels are attributed to their reversible thermal collapse at 37° C.²⁴ To study the effect of temperature-responsive behavior of P(B5AMA) hydrogels on protein refolding efficacies, denatured enzymes were incubated with P-(B5AMA)-5, P(B5AMA)-10, and P(B5AMA)-20 at 37° C., and refolding of lysozyme and carbonic anhydrase was monitored by bacteriolysis of M. lysodeikticus²¹ and by pnitrophenyl acetate (pNPA) hydrolysis,²⁵ respectively (FIGS. 2A and 10). The enzymatic activities of proteins are directly related to the native conformations of protein in physiological conditions, and any changes in the native state of proteins often result in the loss of their enzymatic efficacies: hence, measuring the enzymatic activities of proteins can be used as a parameter to evaluate the protein refolding efficacies of synthetic chaperones.

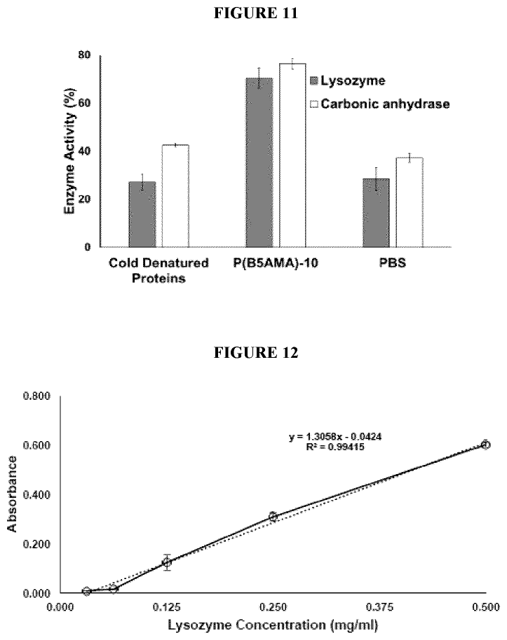

The incubation of heat-denatured proteins in the presence of P(B5AMA) hydrogels at 37° C. exhibited hydrogel crosslinking density-dependent restoration of the enzymatic activity of lysozymes and carbonic anhydrase. The refolding efficacies of lysozyme and carbonic anhydrase in the presence of P(B5AMA)-5 hydrogels were 45±1.64 and 55±1.28, respectively. The increase in the cross-linker concentration of P(B5AMA) hydrogels to 10 mol % improved the enzymatic activity of thermally denatured proteins, and the enzymatic activities of lysozyme and carbonic anhydrase were 65.4±6.58 and 78.65±1.97, respectively, in the presence of P(B5AMA)-10 (FIG. 2A). These potential protein refolding efficacies of P(B5AMA)-10 were not limited to the renaturation of thermally denatured proteins only, but cold-stressed proteins also acquired their biologically active conformation, upon incubation with P(B5AMA)-10 at 37° C. for 3 h (FIG. 11). Further increase in the cross-linker concentration of hydrogels to 20 mol %, however, impaired the chaperone-like activities of P(B5AMA)-20, and the enzymatic activities were comparable to the intrinsic folding efficacies of denatured proteins in aqueous solution (35±3.81%). This reduced activity of enzymes in the presence of P(B5AMA)-20 is possibly due to the limited flexibility of highly cross-linked hydrogels, which may result in partial aggregation of denatured proteins on their surface, hence minimizing the conformational flexibility of proteins and impairing their refolding efficacies.²³

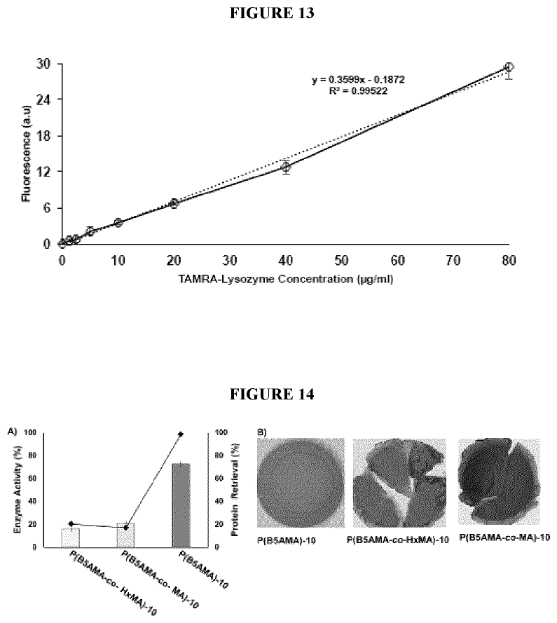

The concentration of enzymes obtained from hydrogel surface was measured by the BCA assay and by quantification of fluorescently labeled proteins incubated in the presence of P(B5AMA)-hydrogels (FIGS. 2, 12 and 13). The results indicated that both native and denatured forms of proteins maintain weak noncovalent interactions with hydrogels of different cross-linker densities and the enzymes can be retrieved from hydrogel surface, in the absence of any external stimuli, such as stripper, temperature, or pH. These weak interactions of proteins with hydrogels are reported by others and are associated with the solvation of a polymeric shell on the hydrogel surface that may minimize the close interactions of proteins with the hydrogel matrix.^{5.23}

Owing to superior thermoresponsive behavior and potential protein refolding efficacies, P(B5AMA)-10 hydrogels were further modified to modulate hydrophobic and ionic interactions with misfolded proteins. P(B5AMA)-10 were synthesized in the presence of hydrophobic and ionic monomers, namely, hexylmethacrylamide (HxMA) and methacrylic acid (MA), respectively, to yield P(B5AMA-co-HxMA)-10 and P(B5AMA-co-MA)-10 hydrogels, and their protein refolding efficacies were evaluated (Table 1 and FIG. 14). Charge-dependent protein refolding efficacies of thermoresponsive nanoparticles have been reported by Shi and co-workers.^{20,21} Their data indicates that thermoresponsive polymeric micelles of both electrostatically repulsive and neutral charges exhibit efficient protein refolding efficacies and optimum hydrophobicity of nanoparticles is a key parameter to maintain weak binding affinity of nanoparticles with denatured proteins, which in turn results in superior refolding of enzymes.^{20,21} The incorporation of hydrophobic and ionic monomers in P(B5AMA)-10 hydrogels, however, showed high retention of proteins in the hydrogel matrix, and their ability to restore enzymatic activities were decreased (FIG. 11). These results suggest that, for P(B5AMA)-10, neutral hydrogels are sufficient to maintain weak nonionic interactions with proteins and to provide the conformational flexibility required for restoration of the enzymatic activity of denatured proteins into the native state.²² The incorporation of ionic and hydrophobic monomers in P(B5AMA)-10 possibly disrupts the balanced interactions of hydrogels with denatured proteins and hence decrease the conformational flexibility of enzymes required for their refolding into the native state.

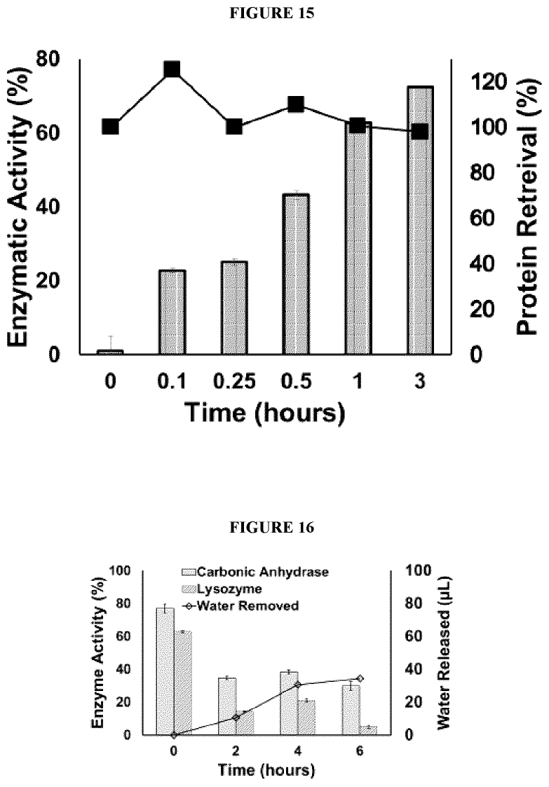

The kinetics of protein refolding in the presence of neutral P(B5AMA)-10 was then studied as a function of temperature and time (FIGS. 2B and 15). Thermally denatured lysozymes were incubated in the presence of P(B5AMA)-10 at different temperatures (4, 25, 37, and 50° C.) and for different time periods (5 min to 24 h), and the enzymatic activity of lysozyme was measured by the bacteriolysis activity of M. lysodeikticus. The results demonstrate that enzymatic activities of lysozyme are optimal at a physiological temperature (37° C.), and inefficient protein activities are obtained at 4, 25, and 50° C. The superior enzymatic activities of proteins in the presence of P(B5AMA)-10 at 37° C. are attributed to the reversible thermal collapse of hydrogels at the specified temperature.²⁴ At a temperature lower than 37° C., hydrogels do not undergo thermal transition: in contrast at higher temperatures, P(B5AMA)-10 exhibit an irreversible collapse of their structures,²⁴ suggesting that reversible structural changes in the hydrogel matrix are important for the optimum protein refolding efficacies of hydrogels at 37° C. (FIGS. 2B and 15). Furthermore, the refolding of enzymes in the presence of P(B5AMA)-10 is a slow process and requires up to 3 h for completion. In comparison to GroEL/GroES chaperone activity, where GroEL takes minutes to cycle between the hydrophilic and hydrophobic states in the presence of ATP and GroES, the slow structural adaptations of synthetic chaperones, as a function of stimuli, are considered responsible for their slow protein refolding efficacies.⁵

To understand the role of cross-linking density of hydrogels in the structural adaptations that may have participated in the restoration of enzymatic activities of proteins, wet and freeze-dried P(B5AMA) hydrogels were first evaluated by SEM for their pore sizes and surface properties (FIG. 3). SEM images reveal that dried P(B5AMA)-5 exhibit a loosely bound meshwork of polymer chains with poorly defined pores, in contrast to P(B5AMA)-10 and P(B5AMA)-20 hydrogels that exhibit well-defined porous structures of uniform density.

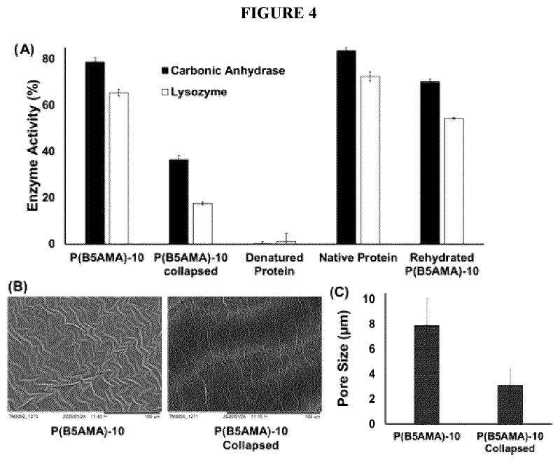

P(B5AMA)-10 and P(B5AMA)-20 showed cross-linker concentration-dependent decreases in the mean pore sizes of hydrogels, and the pore sizes measured were 7.87±2.2 and 5.72±2.1 μm, respectively (FIG. 3C). The hydrated surfaces of P(B5AMA) hydrogels also exhibit a cross-linker concentration-dependent surface roughness. P(B5AMA)-5 showed relatively smooth surfaces, while progressively rough and wrinkled surfaces were observed for P(B5AMA)-10 and P(B5AMA)-20 (FIG. 3A). To understand how the unique features of P(B5AMA)-10 (water release efficacies at 37° C., pore sizes of 7.87±2.2 μm, and relatively wrinkled surface texture) participate in their superior capability to restore the enzymatic activities of proteins at 37° C., P(B5AMA)-10 were incubated at 37° C. to remove the loosely bound water from their matrix, and the enzymatic activities and structural changes in the hydrogel matrix were analyzed (FIG. 4). The data reveals that water release efficacies of P(B5AMA)-10 at 37° C. are directly related to their enzymatic efficacies of proteins (FIG. 16), and collapsed P(B5AMA)-10 (after the removal of free residual water) exhibit significantly reduced enzymatic activities (17.58±0.6 for lysozyme and 36.5±1.8 for carbonic anhydrase) than their hydrated counterparts.

Moreover, this reduced enzymatic activity of proteins was not associated with the entrapment of proteins in collapsed P(B5AMA)-10 hydrogels, and the enzymes maintained their weak noncovalent interactions with both hydrated and collapsed forms of P(B5AMA)-10 hydrogels (Table 4). The residual water-dependent enzymatic activities of proteins in the presence of P-(B5AMA)-10 are intriguing, as protein incubation in the presence of hydrogels is being performed in the presence of excess amount of solvent (buffer) on the hydrogel surface, and the release of a trace amount of water by hydrogels (less than 20 μL) seems to play a dominant role in protein refolding behavior of P(B5AMA)-10.

TABLE 4
Amount of fluorescently labelled protein collected from the surface
of collapsed P(B5AMA)-10, as a function of time was compared with
feed concentration of proteins added to the hydrogel surface.

| Time (minutes | Protein Retrieval (%) |
|---|---|
| 10 | 92.2 ± 3.41 |
| 30 | 93.03 ± 3.33 |
| 60 | 93.69 ± 3.94 |
| 180 | 92.59 ± 2.98 |

A number of experimental and theoretical models indicate that the presence of water in confined geometries plays an important role in the biological activities of macromolecules.^{3,17,26,27} To understand the role of P(B5AMA)-10-confined water in the protein refolding behavior of hydrogels, P(B5AMA)-10 were collapsed at 37° C. and the water collected from hydrogels was freeze-dried and analyzed by ¹H NMR and FTIR for the presence of any impurities that may have participated in the restoration of the enzymatic activities of proteins. However, only a trace amount of free B5AMA (less than 0.03% of the original feed ratio) was detected by ¹H-NMR. The measurement of the enzymatic activities of proteins performed in the presence of a trace amount of free B5AMA and P(B5AMA) hydrogels indicated that free B5AMA do not interfere (aid or hinder) with the enzymatic activities of proteins (data not shown), suggesting that the residual water of B5AMA itself is not directly related to the protein refolding efficacies of P(B5AMA)-10. However, the residual water of P(B5AMA)-10 is documented to facilitate the structural changes in hydrogels as a function of temperature,²⁴ which may have facilitated the restoration of native and biologically active forms of proteins.

In comparison to other thermoresponsive systems (such as PNIPAM and PEGMA), which are solely dependent upon temperature for their oscillation between the hydrophilic and hydrophobic states, P(B5AMA)-10 hydrogels exhibit water content and temperature-dependent oscillatory behavior, and the removal of loosely bound water from the hydrogel architecture above 35° C. locks them into the collapsed state.²⁴ The rehydration of hydrogels to their native form requires both lower temperatures and supply of lost water to the hydrogel matrix.²⁴ To understand the role of structural adaptations of P(B5AMA)-10 in protein refolding efficacies, collapsed P(B5AMA)-10 were rehydrated at room temperature and were re-evaluated for their protein refolding behavior. The rehydration of P(B5AMA)-10 at lower temperatures restored the enzymatic activities of denatured proteins, suggesting that the reduced protein refolding capabilities of collapsed P(B5AMA)-10 at 37° C. were possibly due to the inability of collapsed gels to revert back to their swollen states at high temperatures.

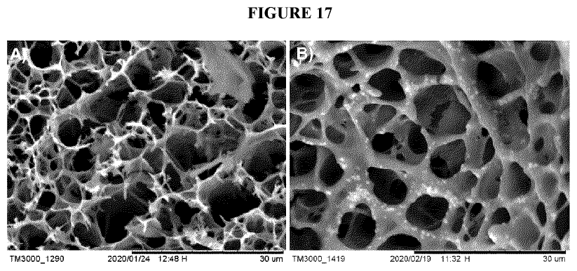

The structural adaptations of P(B5AMA)-10 in both hydrated and collapsed forms were monitored by measuring the changes in the diameter, pore size, and surface texture of hydrogels. SEM images showed that hydrated P(B5AMA)-10 possess relatively smooth surfaces and their freeze-dried forms show a pore size of 7.87±2.2 μm. In contrast, the collapsed state of P(B5AMA-10) showed a highly wrinkled surface texture, and their pore size was reduced to 3.1±1.34 μm (FIGS. 4B, 4C, 17 and Table 5). This reduction in the pore size of P(B5AMA)-10 at the physiological temperature increases the surface roughness of hydrogels, and these structural adaptations of hydrogels are important for their protein refolding efficacies.

TABLE 5
Changes in diameter of P(B5AMA)-10 hydrogel, as a function
of time and water released from the polymeric matrix.

| Time (hr) | Mean Gel Diameter (cm) |
|---|---|
| 0 | 1.30 |
| 2 | 1.24 |
| 4 | 1.21 |
| 6 | 1.20 |
| 24 | 1.20 |

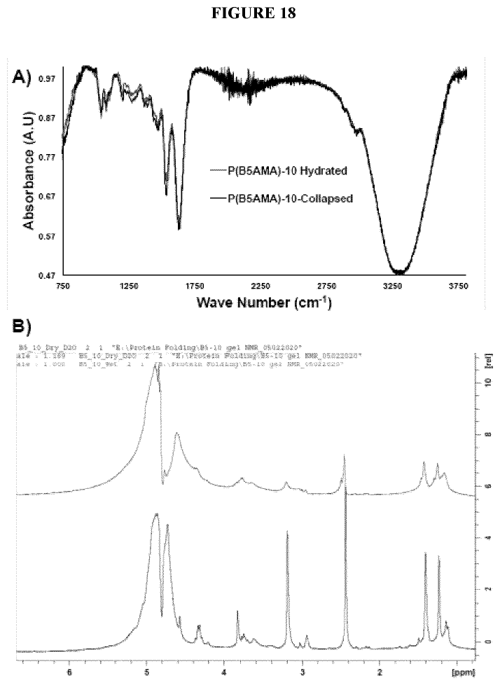

The changes in the chemical compositions of hydrogels at the hydrated and collapsed states, which may have participated in the physical changes of the hydrogel matrix, were further analyzed by ATR-FTIR and ¹H NMR, and the intensity of the functional groups as a function of temperature was recorded. ATR-FTIR analysis of hydrated and collapsed P(B5AMA)-10 exhibited a broad OH group stretching of B5AMA at 3282 cm⁻¹, a vibrational stretching of C═O at 1647 cm⁻¹, a stretching of C—O of alcohols at 1050 cm⁻¹, and a C—N stretch of amides at 1127 cm^{-1.24}, however, there was no significant change in signal intensities or peak shifting of the functional groups in both hydrated and collapsed forms of P(B5AMA)-10. The availability of polar groups of P(B5AMA)-10 in both collapsed and hydrated states is possibly due to a high water content of hydrogels (˜90% of the weight of dried hydrogel matrix) itself, and the release of a trace amount of water at 37° C. (15 μL over a period of 3 h) is not sufficient to detect the changes in signal intensities of the functional groups of hydrogels by FTIR. In contrast, the ¹H-NMR spectra of hydrated hydrogels at 25° C. in D₂O showed significant signal broadening of CH₃ peaks and —CH₂— peaks associated with the B5AMA monomer, indicating the involvement of protons in hydrogen bonding during hydrogelation. The collapsed P(B5AMA)-10 hydrogels at 37° C., in contrast, exhibited characteristic sharp peaks of B5AMA, suggesting that intramolecular noncovalent interactions of B5AMA chains with water molecules are disrupted and B5AMA protons show their characteristic signal in the ¹H-NMR spectrum^{28,28,29} (FIG. 18).

As discussed above, hydrated P(B5AMA)-10 are hygroscopic polymeric materials, which hold more than 90% of their own weight in the form of water, and the presence of residual water in the hydrogel matrix plays a key role in P(B5AMA)-10 thermal flexibility. This can be predicted that the disruption of hydrogen-bonding interactions between P(B5AMA)-10 functional groups and the solvent (water) may disrupt the thermal flexibility of hydrogels, which in turn can alter their protein refolding efficacies.²⁴ To understand the role of hydrogen-bonding interactions in the structural adaptations of P-(B5AMA)-10, the hydrogels were prepared in deuterated water, indicated as dP(B5AMA)-10, and were evaluated for their ability to restore the enzymatic activities of heat-denatured proteins. dP(B5AMA)-10 lost the intrinsic transition behavior of dP(B5AMA)-10 as a function of temperature and exhibited poor enzymatic efficacies of proteins. These results suggest that hydrogen-bonding interactions between P(B5AMA)-10 and entrapped water molecules are important for the thermal flexibility of hydrogels and for their subsequent role in protein refolding efficacies.

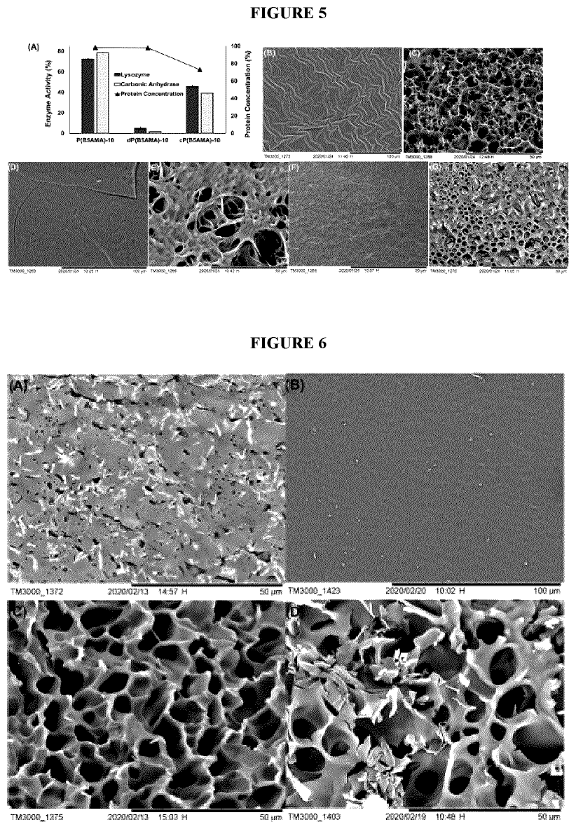

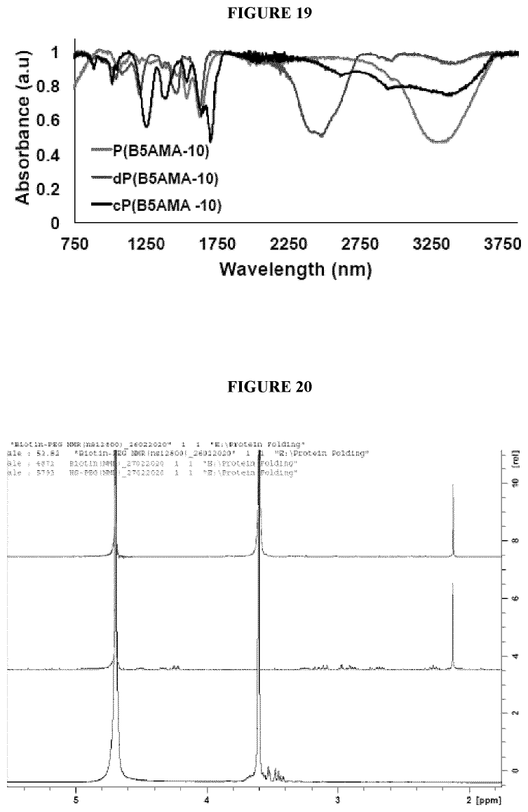

The reduced hydrogen-bonding interactions between polymer chains of P(B5AMA) hydrogel and with solvent molecules were then confirmed by FTIR spectroscopy. The OH group stretching of P(B5AMA)-10 at 3282 cm⁻¹ was significantly diminished in dP(B5AMA)-10, and a characteristic O-D stretching of deuterated water at 2480 cm⁻¹ was observed,³⁰ indicating the entrapment of D₂O in porous P(B5AMA) matrix. The reduced hydrogen-bonding interactions in the dP(B5AMA)-10 matrix also translated into their poor thermal flexibility and reduced capabilities to restore the enzymatic activities of denatured proteins. The role of hydrogen-bonding interactions of P(B5AMA)-10 in protein refolding efficacies was further evaluated by the acetyl capping of the hydroxyl group of P(B5AMA)-10, and the enzymatic activities of proteins in the presence of an acetyl group-capped P(B5AMA)-10 (indicated as cP(B5AMA)-10) were evaluated (FIGS. 5A and 19). The acetyl capping of P(B5AMA)-10 post treatment with acetic anhydride was found to be successful by FTIR spectroscopy. cP(B5AMA)-10 showed a characteristic C—O stretch of esters at 1250 cm⁻¹, bending and stretching of alkanes at 1350 and 2920 cm⁻¹, respectively, and a C═O stretch at 1710 cm^{-1.31} Moreover, the disruption of hydrogen-bonding interaction of the cP(B5AMA)-10 matrix was apparent by the reduced OH group stretching of P(B5AMA)-10 at 3282 cm⁻¹;however, the signal intensity of the OH group stretching was stronger than that of dP(B5AMA)-10, suggesting the presence of water molecules in the cP(B5AMA)-10 matrix.

cP(B5AMA)-10 also showed impaired temperature-responsive behavior of hydrogels, and poor activities of enzymes at 37° C. were obtained. The reduced capabilities of cP(B5AMA)-10 to restore the enzymatic activities of heat-denatured proteins were not associated with the increase in the hydrophobicity of hydrogels, and proteins maintained their non-ionic and weak interactions with the hydrogel matrix, suggesting that the thermal flexibility of P(B5AMA)-10 is important for their efficient protein refolding efficacies. SEM images of wet and dried samples of hydrogels further illustrated that the disruption of hydrogen bonding in the polymer matrix either by acetyl capping or by the solvent exchange altered the well-defined porous structure of native P(B5AMA)-10, and distorted pores of non-uniform sizes were observed. Moreover, wet surfaces of cP(B5AMA)-10 and dP(B5AMA)-10 lost the distinct wrinkle-like morphology of P(B5AMA)-10, suggesting that hydrogen bonding between water molecules and P(B5AMA)-10 is important for unique thermally flexible structure of hydrogels, and the disruption of noncovalent interactions between water molecules and hydrogel matrix significantly impacts their functional applications (protein refolding efficacies and water release behavior) (FIG. 5).

The distinguishing properties of P(B5AMA)-10 hydrogels are their textured surface, well-defined micron-sized pores in their architecture, and their capability to change the surface roughness and pore sizes as a function of temperature. To evaluate if the confinement of denatured proteins in the porous structure of P(B5AMA)-10 or surface adaptations of hydrogels as a function of temperature are responsible for their superior protein refolding efficacies, streptavidin was used as a model protein and streptavidin-biotin interactions were employed to detect the presence of proteins in the hydrogel matrix. For this purpose, streptavidin-containing hydrogel matrix was labeled with biotinylated gold nanoparticles, and the presence of streptavidin either on the surface of hydrogels or inside the pores was probed by SEM (FIGS. 6, 20 and Table 6). SEM images reveal that in the absence of streptavidin only a small amount of biotinylated GNPs are present on the surface of P(B5AMA)-10; in contrast, streptavidin-incubated hydrogels showed a remarkable accumulation of biotinylated GNPs on their surface. Furthermore, analysis of the porous structure of P(B5AMA)-10 revealed a negligible amount of biotinylated GNPs in both the presence and absence of streptavidin, suggesting that denatured enzymes likely interact with the surface of P(B5AMA)-10 and the structural adaptations of P(B5AMA)-10 surface as a function of temperature participate in protein refolding efficacies. The changes in pore sizes of P(B5AMA)-10 at different temperatures are inarguably important to yield thermal flexibility to the hydrogel surface; however, the confinement of proteins in the hydrogel pore does not seem to participate in protein refolding. These results are also in agreement with the recent literature, where fast relaxation imaging technique was utilized to evaluate the thermal stability of proteins on poly(acrylamide) hydrogel surface, and indicated that the hydrogel surface rather than pore size is important for its interactions with proteins.²³

TABLE 6
Characterization of biotinylated GNPs by dynamic light scattering
and zeta potential instrument and UV absorbance.

| Sample | DLS (nm) | Zeta Potential (mV) | λₘₐₓ (nm) |
|---|---|---|---|
| Citrate capped GNPs | 50.6 ± 0.13 | −34.5 | 527 |
| Biotinylated GNPs | 66.1 ± 0.1 | −17.4 | 534 |

### CONCLUSION

A handful of studies on temperature-responsive hydrogels indicate that these smart hydrogels participate in protein refolding efficacies, as a function of temperature, while maintaining weak and noncovalent interactions with denatured proteins. However, the details of the structural events that may participate during this process are currently unknown. In this study, the role of hydrogels in the restoration of enzymatic activities of heat-denatured proteins, using thermally flexible P(B5AMA) hydrogels is elucidated.

P(B5AMA) hydrogels are smart materials, which exhibit temperature-responsive behavior due to the hydrogen-bonding interactions between water molecules and polymer chains in the hydrogel matrix, and these interactions be tuned as a function of the hydrogel cross-linking density. By utilizing this property of hydrogels, thermally responsive P(B5AMA)-10 hydrogels were trapped in hydrated and collapsed states at different temperatures, and structural changes in the polymeric matrix were correlated with their ability to restore the enzymatic activities of unfolded proteins. Poly(B5AMA)-10 with exceptional thermal flexibility demonstrated significant changes in their surface roughness and pore sizes as a function of temperature, and these physical changes in the hydrogel architecture further translated into their higher protein refolding efficacies, as suggested by the improved enzymatic activities of proteins. The disruption in hydrogen-bonding interactions of the polymeric matrix of hydrogels reduced the thermal flexibility of hydrogels that also resulted in their impaired protein refolding efficacies. Furthermore, denatured proteins were found to weakly interact with the hydrogel surface, and the role of confinement of protein in the porous structure of hydrogels was minimal. Further studies are being conducted to evaluate the role of hydrogels in the restoration of the native structure of amyloid plaques for the treatment of Alzheimer's disease and to confirm the changes in the structure of proteins as a function of the hydrogel flexibility by circular dichroism (CD) spectroscopy.

### EXPERIMENTAL PROCEDURES

Materials. Triethylamine (TEA), anhydrous methanol, potassium persulfate (KPS), N,N,N′,N′-tetramethylethylenediamine (TEMEDA), streptavidin and N,N′-methylene bisacrylamide, potassium phosphate dibasic, sodium phosphate dibasic hexahydrate, Micrococcus lysodeikticus, methacrylic acid, methanol, brilliant blue G, and D-pantolactone were purchased from Sigma-Aldrich. Acetone, diethyl ether, biotin-NHS, 5,6-carboxytetramethylrhodamine succinimidyl ester (TAMRA-NHS), carbonic anhydrase, potassium dihydrogen phosphate, and 4-nitrophenyl acetate sodium phosphate monobasic were obtained from Fisher Scientific. Lysozyme from chicken egg white was purchased from Bio Basic. Gold nanoparticles were purchased. HS-PEG-NH₂ was purchased from NanoCS. 2-Aminoethyl methacrylamide (AEMA) was synthesized according to the previously established procedure.²⁴

Synthesis of B5AMA. B5AMA monomer was synthesized according to the previously established procedure.²⁴ Briefly, 23 mmoles of AEMA was dissolved in anhydrous methanol (4 mL) in the presence of triethylamine (20 mL) under an inert atmosphere, and the mixture was stirred for 3 h at room temperature to obtain a homogeneous solution. Twenty mmoles of pantolactone was then added, and the reaction was allowed to proceed overnight under inert conditions at room temperature. The solution was precipitated in acetone, and the white precipitates of triethylamine hydrochloride were filtered. The B5AMA monomer was purified from the filtrate by silica column chromatography, using acetone as an eluent. The eluent was concentrated and was precipitated in diethyl ether to obtain the final product in the form of a pure yellowish oil with a 65% yield. The synthesis of monomer was confirmed by a Bruker 300 MHz proton nuclear magnetic resonance (¹H NMR) (FIG. 8).

Synthesis of P(B5AMA) Hydrogels. P(B5AMA) hydrogels were prepared by free-radical polymerization method. Briefly, the monomer B5AMA (0.78 M), initiator KPS (10 mM), and the cross-linker N,N′-methylene bisacrylamide (Bis) (5-20 mol %) were dissolved in distilled deionized water, and the solution was degassed for 5-10 min, followed by the addition of 20 μL of TEMEDA (6.6 μM), as catalyst. The hydrogels were formed in 15 min, and gel formation was confirmed by vial inversion test. The hydrogels were repeatedly washed with deionized water to remove any residual monomers and reagents. The percent yield of hydrogels was calculated using the following equation:

$$\% yield of P ( B 5 AMA ) hydrogels = ( initial weight of reactants used for hydrogels synthesis - final weight of freeze dried hydrogel ) / initial weight of reactants used for hydrogels synthesis$$

Synthesis of deuterated B5AMA hydrogels dP(B5AMA-10) was achieved with 10 mol % cross-linker concentration as described above, with D₂O, as a solvent.

Temperature-Dependent Collapse of P(B5AMA-10). P-(B5AMA) hydrogels were incubated at 37° C. for different time periods (5 min to 24 h), and the amount of water released from hydrogels was collected. The collapsed hydrogels were tested for their protein refolding efficacies and, as a function of water, were removed from the hydrogel matrix. The collapsed hydrogels were freeze-dried and analyzed for their pore size by SEM. To determine the leaching of the B5AMA monomer from the hydrogels, P(B5AMA)-10 was incubated at 37° C. and the amount of water released was collected after 24 h. The water released was freeze-dried, and the residual solid was dissolved in deuterium oxide (D₂O) for analysis by ¹H NMR. The methacrylamide peak area of B5AMA at a chemical shift of 5.6862 ppm was integrated and compared with the peak area of standardized B5AMA solutions in D₂O. Using this procedure, the estimated concentration of the residual B5AMA in the released water was found to be less than 0.003% of the monomer feed ratio.

Determination of Hydrogel Pore Size. The freeze-dried hydrogels were imaged using scanning electron microscopy (SEM), and pore sizes of P(B5AMA) hydrogels were analyzed using image J software of version 1.52. A minimum of 100 pores per sample were analyzed, and the data was averaged to determine the mean and standard deviation. SEM micrographs of wet and dried hydrogels were obtained using a Hitachi TM3000 Benchtop Scanning Electron Microscope instrument. All samples were sputter-coated with 60:40 Au/Pd prior to imaging using an SPI-Module Sputter Coater (SPI Supplies).

Acetyl Capping of P(B5AMA)-10. Acetyl group capping of hydrogels cP(B5AMA-10) was achieved by submerging P(B5AMA)-10 in 1 mL acetic anhydride (97%) solution overnight. The modified hydrogels were then washed extensively with deionized water and were submerged in deionized water overnight to remove free acetic anhydride. The acetyl capping of hydrogels was confirmed by attenuated total reflectance-Fourier transform infrared (ATR-FTIR) spectrophotometer. The surface roughness and pore sizes of hydrogels were determined by SEM (Hitachi TM3000 Benchtop Scanning Electron Microscope instrument). ATR-FTIR was performed on a Bruker Alpha-T single reflection attenuated ATR module equipped with a platinum-diamond crystal. Spectra were collected from 16 scans per sample and subtracted from background spectra, between 375 and 4000 cm⁻¹ at a resolution of 0.9 cm⁻¹.

Heat Denaturation of Enzymes. Lysozyme was dissolved in 50 mM potassium phosphate buffer (pH 6.24) at a concentration of 1600 units/mL (80 μg/mL). Lysozyme containing aqueous solution was incubated at 110° C. for 5 h. M. lysodeikticus suspension (0.15 mg/mL) was prepared by using 50 mM potassium phosphate buffer (pH 6.24), and 100 μL of heat-denatured or native lysozyme was added to 900 μL of M. lysodeikticus suspension. The lysosomal activity was recorded by measuring the sample absorbance at 450 nm for 5 min using UV-vis spectrophotometer (Hewlett Packard, HP8453). The aqueous solution of carbonic anhydrase (75 μg/mL) was prepared in 20 mM Tris-sulfate buffer (pH 7.75), and the solution was denatured at 110° C. for 5 h. Nine hundred and ninety microliters of native or denatured carbonic anhydrase solution was then mixed with 10 μL of 52 mM 4-nitrophenyl acetate (NPA), and the absorbance of the solution was recorded at 400 nm after 5 min, using UV-vis spectrophotometry (Hewlett Packard, HP8453). Denaturation of native lysozyme and carbonic anhydrase and the formation of aggregates at 110° C. were studied by ATR-FTIR on a Bruker Alpha-T single reflection attenuated ATR module equipped with a platinum-diamond crystal. Spectra were collected from 16 scans per sample and subtracted from background spectra, between 375 and 4000 cm⁻¹ at a resolution of 0.9 cm⁻¹ and by dynamic light scattering (DLS) and a ζ-potential instrument (Brookhaven 90 Plus PALS).

Cold Denaturation of Enzymes. Lysozyme (1600 units/mL) and carbonic anhydrase (75 μg/mL) were denatured in liquid nitrogen (−80° C.) for 10 min and were thawed at room temperature for 15 min. The denaturation of enzymes was confirmed by the bacteriolysis capability of lysozyme and by the NPA hydrolysis assay of carbonic anhydrase, as described above.

Enzymatic Activities of Lysozyme and Carbonic Anhydrase in the Presence of P(B5AMA) Hydrogels. Heat-and cold-denatured lysozyme (80 μg/mL) and carbonic anhydrase (75 μg/mL) were incubated in the presence of P(B5AMA) hydrogels at different temperatures (4° C., room temperature, 37° C., and 50° C.) and for different time periods (5 min to 24 h). The enzymatic activities of native and denatured lysozymes incubated in the presence of hydrogels were tested by the bacteriolysis capability of lysozyme and by the NPA hydrolysis assay of carbonic anhydrase, as described above. All of the experiments were performed in triplicates and were repeated at least twice to confirm the repeatability of data.

Enzyme Quantification Assays. Bicinchoninic Acid Assay (BCA). Denatured and native lysozymes were incubated with P(B5AMA) hydrogels for different time intervals (5 min to 24 h) and at different temperatures (4° C., room temperature, 37° C., and 50° C.). The aliquots of enzymes were collected at different time points, and the amount of protein in the solution was measured by the BCA protein assay kit (Pierce), according to the manufacturer's protocol. The amount of protein retrieved from the hydrogel solution was determined using the calibration curve of lysozyme as the standard, with a working range of 20-2000 μg/mL of lysozyme.

Fluorescent Labeling of Enzymes. Seventy micrometers of lysozyme solutions were prepared in a phosphate-buffered saline (PBS) of pH 6.24 and was incubated with 10 μL of 7 mM TAMRANHS stock solution in DMSO, under constant stirring in dark at room temperature for 2 h. TAMRA-labeled lysozyme was purified using 5 kDa microcentrifuge filters, and the labeled protein was freeze-dried to achieve pure conjugates.

Quantification of Fluorescently Labeled Lysozymes. TAMRA-labeled lysozyme (80 μg/mL) was incubated with P-(B5AMA) hydrogels at different temperatures (4° C., rt, 37° C., and 50° C.). Hundred microliters of aliquots were collected from fluorescent samples incubated in the presence of P(B5AMA) hydrogels at different time intervals (5 min to 24 h). The amount of protein in the solution was evaluated by exciting TAMRA-labeled protein at 550 nm and by measuring its emission at 580 nm, using a Varioskan LUX multimode microplate reader. The amount of protein in the solution was determined by using a calibration curve of TAMRA-labeled lysozyme as a control.

Coomassie Blue Staining of Enzyme-Incubated Hydrogels. Heat-denatured lysozyme (1600 units/mL) was incubated with P(B5AMA) hydrogels for 3 h. The supernatant was removed, and the hydrogels were fixed for 1 h in a methanol/acetic acid (5:1 vol/vol ratio) mixture to prevent the loss of the attached protein upon staining. Fixed P(B5AMA) hydrogels were incubated with a 11.7 μM Brilliant Blue G solution for 1 h. The stained hydrogels were destained in an acetic acid/methanol/water (50:40:10) mixture overnight and were imaged using a BluPAD dual LED blue/white light trans-illuminator.

Interactions of Proteins with P(B5AMA) Hydrogels. Streptavidin-biotin interactions were utilized to determine the interactions of protein with P(B5AMA)-10 hydrogel matrix. Biotin-functionalized gold nanoparticles (GNPs) were prepared in two steps: In the first step, 50 μL of 4.51 mM biotin-NHS was reacted with 500 μL of 0.2 mM HS-PEG-NH2 in the PBS of pH 6.24 for 4 h at room temperature. The mixture was purified to remove free biotin, and biotin-PEG-SH (250 μL) was added to 50 nm of gold colloids (500 μL) in the presence of 500 μL of mPEG-SH (0.2 mM), and the mixture was stirred for 4 h at room temperature. The nanoparticles were centrifuged, washed three times with deionized water, and dispersed in deionized water. The hydrodynamic diameter and the net charge of gold nanoparticles were measured by dynamic light scattering (DLS) and a ζ-potential instrument (Brookhaven 90 Plus PALS).

In the second step, streptavidin was incubated with P(B5AMA) hydrogels for 3 h, and streptavidin-incubated hydrogels were fixed in a methanol/acetic acid mixture (5:1 vol/vol ratio) for 60 min. The supernatant was then removed, and streptavidin-fixed P(B5AMA) hydrogels were washed with deionized water and incubated with biotinylated GNPs at room temperature for 4 h. The hydrogels were then washed to remove any free gold nanoparticles, freeze-dried, and imaged using the SEM imaging technique. P(B5AMA) hydrogels in the absence of streptavidin were used as a control to evaluate the nonspecific interactions of biotinylated GNPs with hydrogels.

### Example 2: Improving Storage Stability of Protein Therapeutics Using Hydrophilic Hydrogels

Example 1 details the development of stimuli responsive hydrogels that can stabilize thermally denatured enzymes at a concentration of to 1.3 mg/m² of hydrogels and prevent their aggregation in solution form. Poly(B5AMA) hydrogels are also extremely hygroscopic, can serve as an excellent source for dewatering of sensitive biomolecules such as proteins and peptide based therapeutics and their protein refolding efficacies are directly linked to their water desorption capabilities. Poly(B5AMA) hydrogels act as macromolecular chaperones and show exceptional capabilities to fold thermally denatured proteins, as a function of temperature. In this Example, application of the water absorption and protein refolding capabilities of poly(B5AMA) hydrogels to enhance the storage stability of therapeutic proteins (enzymes, antibodies and cytokines) in both liquid and solid forms will be shown.

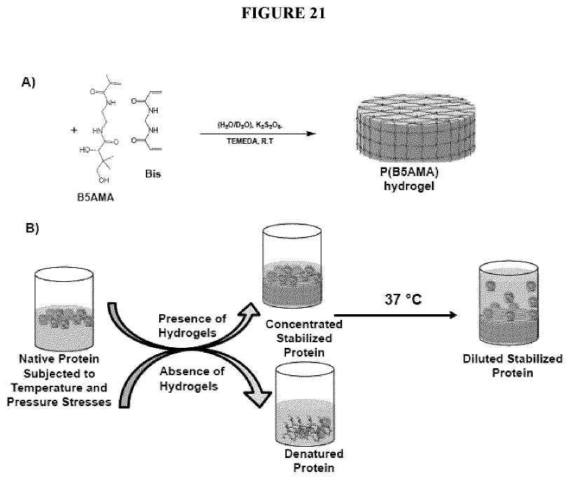

Utilizing the water absorption/desorption and protein refolding efficacies of poly(B5AMA) hydrogels, this Example will apply the hydrogels to improve the storage stability and shelf life of therapeutic proteins. Poly(B5AMA) hydrogels may absorb 6.8 g of water per gram of hydrogel and may stabilize and refold 1.3 mg of thermally denatured polypeptide per m² of hydrogels.³⁹ Using this previous knowledge, temperature responsive poly(B5AMA) hydrogels will be synthesized by free radical polymerization method (FIG. 21). Efficacies of temperature responsive hydrogels in dewatering of different therapeutic protein (antibodies, enzymes, and cytokines) and in maintaining their physical, chemical and biological stabilities will be evaluated.

Protein-based therapeutics can be broadly divided into monoclonal antibodies, cytokines and enzymes. Monoclonal antibodies (mAbs) are immuno-globular proteins of ˜150 kDa, which are used as antigen binders for diagnostic and for cancer therapy. At present at least 29 mAbs and their drug conjugates are approved for clinical uses and are among one of the top selling proteins across the globe.⁴⁴

Antibodies are typically administered at high concentrations (20-120 mg/mL) for cancer therapy, hence their solution stability is important for their facile applications in clinical settings.

At present, stability of antibodies in solution form is aided with the help of excipients (such as electrolytes, polyols and non-ionic detergents)⁴⁵, however presence of excipients in protein solutions can cause adverse reactions and decrease the efficacies of therapeutic proteins in certain patients.⁴⁶

Cytokines are glycoproteins of less than 30 kDa, which act as modulators of immune response by controlling growth activation and differentiation of various cell types. Production of cytokines includes a number of processes including purification, filtration, transportation, and storage, which can perturb native structure of proteins, causing loss of their activity.³⁹⁻⁴⁰ Furthermore, cytokines are generally administered at low doses and have narrow therapeutic index and thus structural stability is a key factor in ensuring their efficacies. Another major issue with cytokine based therapeutics is their adsorption on storage containers and difficulties in assessing their structural integrity in the presence of excipients.

Enzymes are proteins of different sizes with capabilities to revolutionize chemical and bio-pharmaceutical industries, however enzymes are prone to degradation in repose to common stresses such as transport, storage upon desiccation, freeze thawing and freeze drying.⁴¹⁻⁴⁴

In this Example, various types of proteins of different sizes, hydrophobicity and of clinical administration conditions will be evaluated for their stability in response to temperature stress, in the presence of poly(B5AMA) hydrogels. IL-10, IL-6, and TNF-α were selected as representative cytokines and lysozyme was selected as a representative enzyme.

### Evaluation of Freeze-thawing Cytokines in the Presence and Absence of Poly(B5AMA)

An 80 ng/ml solution of TNF-α in a 0.1 M carbonate-bicarbonate buffer (pH 9.6) and a 400 ng/mL solution of IL-10 using the same carbonate-bicarbonate buffer were prepared. Portions of each of the solutions were then added over poly(B5AMA) hydrogels at an approximate ratio of about 19 mL of solution per gram of dry hydrogel.

The solutions (alone and the portions added over the hydrogels) were frozen at −20° C. for at least 16 hours and subsequently thawed. The cytokine activities of the solutions were then determined using an ELISA assay. The resulting cytokine activities of the solutions were then compared to the activities of the native cytokines.

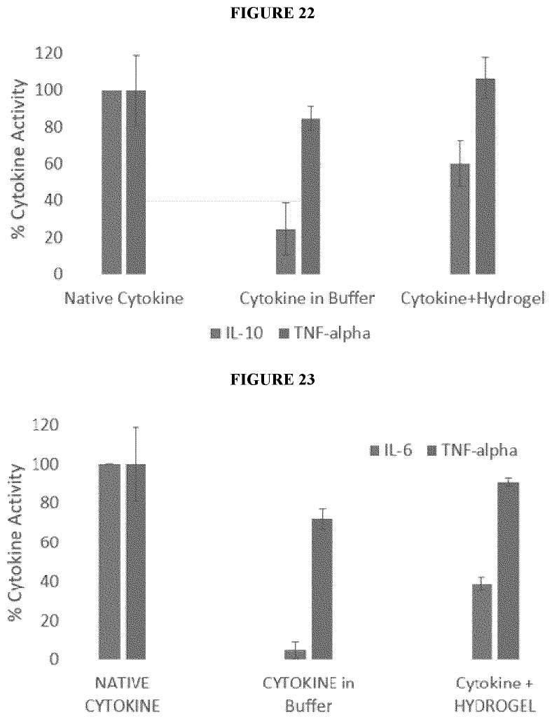

The results are shown in FIG. 22, wherein the red bars show the cytokine activities of native TNF-α and TNF-α solutions, and the blue bars show the cytokine activities of native IL-10 and IL-10 solutions.

As shown, the negative impact of freeze-thawing on cytokine activity is significantly reduced when the solutions added over the poly(B5AMA) hydrogel. In more detail, the cytokine activity of the TNF-α solution that was not added over the poly(B5AMA) hydrogel was reduced to about 80%, while the cytokine activity of the TNF-α solution that was added over the poly(B5AMA) hydrogel was maintained at about 100%. As well, the cytokine activity of the IL-10 solution that was not added over the poly(B5AMA) hydrogel was reduced to about 25%, while the cytokine activity of the IL-10 solution that was added over the poly(B5AMA) hydrogel was reduced only to about 60%.

### Evaluation of Freeze-drying Cytokines in the Presence and Absence of Poly(B5AMA)

An 80 ng/ml solution of TNF-α in a 0.1 M carbonate-bicarbonate buffer (pH 9.6) and a 400 ng/mL solution of IL-10 using the same carbonate-bicarbonate buffer were prepared. Portions of each of the solutions were then added over poly(B5AMA) hydrogels at an approximate ratio of about 19 mL of solution per gram of dry hydrogel.

The solutions (alone and the portions added over the hydrogels) were freeze-dried at a temperature of −50° C. and a pressure of 0.1 mbar and for at least 16 hours. After freeze-drying, the hydrogels and the solutions added thereover were reconstituted with distilled water to maintain a final concentration of 0.1M carbonate-bicarbonate buffer. Then, the cytokine activities of the solutions were determined using an ELISA assay. The resulting cytokine activities of the solutions were then compared to the activities of the native cytokines.

The results are shown in FIG. 23, wherein the red bars show the cytokine activities of native TNF-α and TNF-α solutions, and the blue bars show the cytokine activities of native IL-6 and IL-6 solutions.

As shown, the negative impact of freeze-thawing on cytokine activity is significantly reduced when the solutions are added over the poly(B5AMA) hydrogel. In more detail, the cytokine activity of the TNF-α solution not added over the poly(B5AMA) hydrogel was reduced to about 75%, while the cytokine activity of the TNF-α solution added over the poly(B5AMA) hydrogel was reduced only to about 95%. As well, the cytokine activity of the IL-6 solution not added over the poly(B5AMA) hydrogel was reduced to about 5%, while the cytokine activity of the IL-6 solution added over the poly(B5AMA) hydrogel was reduced only to about 40%.

### Evaluation of Enzyme Activity and Concentration during Heat Denaturation

85 microgram/mL lysozyme in 50 mM phosphate buffer (pH 6.24) solutions were prepared and subjected to heat denaturation. One solution was subjected to the heat denaturation in the presence of a poly(B5AMA) hydrogel and one without.

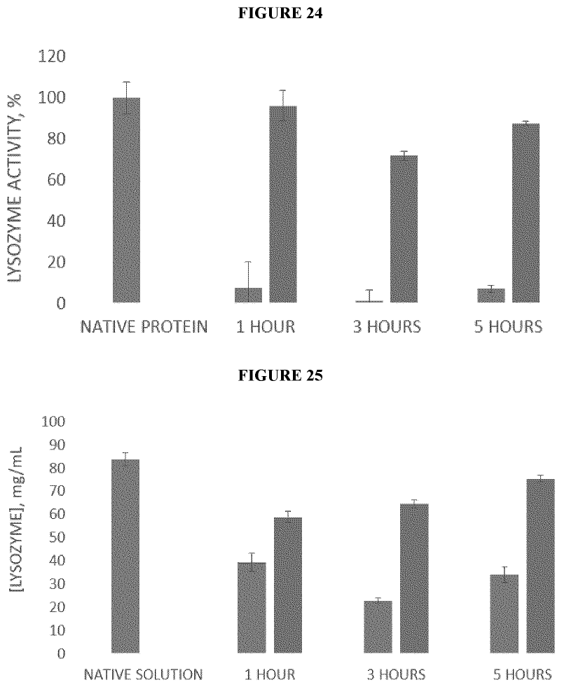

The heat denaturation involved heating the lysozyme solutions at a temperature of 80° C. for a period of 5 hours. Over the course of the 5 hours, the lysozyme activity and concentration of the solutions were monitored. Lysozyme activity was determined using Micrococcus lysodeikticus as a substrate. As will be understood, live bacterial cells yield turbid solutions that strongly absorb at 600 nm. Lysozyme acts on the wall of Micrococcus lysodeikitcus, killing the bacteria and reducing the turbidity of the bacterial sample. This change in absorbance is recorded at 600 nm and indicates the bacterial activity. The results are shown in FIG. 24 and FIG. 25.

FIG. 24 shows the change in lysozyme activity of the solutions over the course of the 5 hour heat denaturation as compared to the native protein, wherein the blue bars represent the lysozyme activity of the native protein and the lysozyme solution denatured in the absence of the poly(B5AMA) hydrogel, and the red bars represent the lysozyme activity of the lysozyme solution denatured in the presence of the poly(B5AMA hydrogel). As shown, the lysozyme activity of the solution denatured in the absence of the poly(B5AMA) hydrogel was reduced to about 5% after the 5 hours, whereas the lysozyme activity of the solution denatured in the presence of the poly(B5AMA) hydrogel was reduced only to about 90% after the 5 hours.

FIG. 25 shows the change in lysozyme concentration of the solutions over the course of the 5 hour heat denaturation as compared to the starting concentration, wherein the blue bars represent the lysozyme concentration of the initial solutions and the lysozyme solution denatured in the absence of the poly(B5AMA) hydrogel, and the red bars represent the lysozyme concentration of the lysozyme solution denatured in the presence of the poly(B5AMA hydrogel). As shown, the lysozyme concentration of the solution denatured in the absence of the poly(B5AMA) hydrogel was reduced to about 35% after the 5 hours, whereas the lysozyme concentration of the solution denatured in the presence of the poly(B5AMA) hydrogel was reduced only to about 75% after the 5 hours.

### CONCLUSION

As shown from the above results, poly(B5AMA) hydrogels may be effective at maintaining protein activity and concentration after subjection to common laboratory processes such as freeze-thawing, freeze-drying, and heat treatments.

### REFERENCES

(1) Dandage, R.; Bandyopadhyay, A.; Jayaraj, G. G.; Saxena, K.; Dalal, V.; Das, A.; Chakraborty, K. Classification of Chemical Chaperones Based on Their Effect on Protein Folding Landscapes. ACS Chem. Biol. 2015, 10, 813-820.

(2) Pradhan, N.; Debnath, K.; Mandal, S.; Jana, N. R.; Jana, N. R. Antiamyloidogenic Chemical/Biochemical-Based Designed Nanoparticle as Artificial Chaperone for Efficient Inhibition of Protein Aggregation. Biomacromolecules 2018, 19, 1721-1731.

(3) Liu, H.; Dong, X.; Sun, Y. Enhanced Protein Adsorption and Facilitated Refolding of Like-Charged Protein with Highly Charged Silica Nanoparticles Fabricated by Sequential Double Modifications. Langmuir 2015, 31, 655-658.

(4) Cavalieri, F.; Chiessi, E.; Paradossi, G. Chaperone-like Activity of Nanoparticles of Hydrophobized Poly(Vinyl Alcohol). Soft Matter 2007, 3, 718-724.

(5) Jones, H.; Dalmaris, J.; Wright, M.; Steinke, J. H. G.; Miller, A. D. Hydrogel Polymer Appears To Mimic The Performance Of The GroEL/GroES Molecular Chaperone Machine. Org. Biomol. Chem. 2006, 4, 2568-2574.

(6) Morimoto, N.; Endo, T.; Iwasaki, Y.; Akiyoshi, K. Design of Hybrid Hydrogels with Self-Assembled Nanogels as Cross-Linkers: Interaction with Proteins and Chaperone-Like Activity. Biomacromolecules 2005, 6, 1829-1834.

(7) Nomura, Y.; Ikeda, M.; Yamaguchi, N.; Aoyama, Y.; Akiyoshi, K. Protein Refolding Assisted By Self-Assembled Nanogels As Novel Artificial Molecular Chaperone. FEBS Lett. 2003, 553, 271-276.

(8) Lu, D.; Zhang, K.; Liu, Z. Protein Refolding Assisted By An Artificial Chaperone Using Temperature Stimuli Responsive Polymer As The Stripper. Biochem. Eng. J. 2005, 25, 141-149.

(9) Fujita, K.; Nakano, R.; Nakaba, R.; Nakamura, N.; Ohno, H. Hydrated Ionic Liquids Enable Both Solubilisation and Refolding of Aggregated Concanavalin A. Chem. Commun. 2019, 55, 3578-3581.

(10) Machida, S.; Ogawa, S.; Xiaohua, S.; Takaha, T.; Fujii, K.; Hayashi, K. Cycloamylose as an Efficient Artificial Chaperone For Protein Refolding. FEBS Lett. 2000, 486, 131-135.

(11) Christiansen, A.; Wang, Q.; Cheung, M.; Wittung-Stafshede, P. Effects of Macromolecular Crowding Agents on Protein Folding In Vitro And In Silico. Biophys. Rev. 2013, 5, 137-145.

(12) Baker, S. L.; Munasinghe, A.; Murata, H.; Lin, P.; Matyjaszewski, K.; Colina, C. M.; Russell, A. J. Intramolecular Interactions of Conjugated Polymers Mimic Molecular Chaperones to Stabilize Protein-Polymer Conjugates. Biomacromolecules 2018, 19, 3798-3813.

(13) Pradhan, N.; Shekhar, S.; Jana, N. R.; Jana, N. R. Sugar-Terminated Nanoparticle Chaperones Are 102-105 Times Better Than Molecular Sugars in Inhibiting Protein Aggregation and Reducing Amyloidogenic Cytotoxicity. ACS Appl. Mater. Interfaces 2017, 9, 10554-10566.

(14) Mondal, R.; Ghosh, N.; Paul, B. K.; Mukherjee, S. Triblock-Copolymer-Assisted Mixed-Micelle Formation Results in the Refolding of Unfolded Protein. Langmuir 2018, 34, 896-903.

(15) Nakamoto, M.; Nonaka, T.; Shea, K. J.; Miura, Y.; Hoshino, Y. Design of Synthetic Polymer Nanoparticles That Facilitate Resolubilization and Refolding of Aggregated Positively Charged Lysozyme. J. Am. Chem. Soc. 2016, 138, 4282-4285.

(16) Baker, S. L.; Munasinghe, A.; Murata, H.; Lin, P.; Matyjaszewski, K.; Colina, C. M.; Russell, A. J. Intramolecular Interactions of Conjugated Polymers Mimic Molecular Chaperones to Stabilize Protein-Polymer Conjugates. Biomacromolecules 2018, 19, 3798-3813.

(17) Kameta, N.; Masuda, M.; Shimizu, T. Soft Nanotube Hydrogels Functioning As Artificial Chaperones. ACS Nano 2012, 6, 5249-5258.

(18) Ravindra, R.; Zhao, S.; Gies, H.; Winter, R. Protein Encapsulation in Mesoporous Silicate: The Effects of Confinement on Protein Stability, Hydration, and Volumetric Properties. J. Am. Chem. Soc. 2004, 126, 12224-12225.

(19) Raghava, S.; Singh, P. K.; Ranga Rao, A.; Dutta, V.; Gupta, M. N. Nanoparticles of Unmodified Titanium Dioxide Facilitate Protein Refolding. J. Mater. Chem. 2009, 19, 2830.

(20) Ma, F.; An, Y.; Wang, J.; Song, Y.; Liu, Y.; Shi, L. Synthetic Nanochaperones Facilitate Refolding of Denatured Proteins. ACS Nano 2017, 11, 10549-10557.

(21) Huang, F.; Shen, L.; Wang, J.; Qu, A.; Yang, H.; Zhang, Z.; An, Y.; Shi, L. Effect of the Surface Charge of Artificial Chaperones on the Refolding of Thermally Denatured Lysozymes. ACS Appl. Mater. Interfaces, 2016, 8, 3669-3678.

(22) Liu, X.; Liu, Y.; Zhang, Z.; Huang, F.; Tao, Q.; Ma, R.; An, Y.; Shi, L. Temperature-Responsive Mixed-Shell Polymeric Micelles for the Refolding of Thermally Denatured Proteins. Chem.—Eur. J. 2013, 19, 7437-7442.

(23) Kisley, L.; Serrano, K. A.; Guin, D.; Kong, X.; Gruebele, M.; Leckband, D. E. Direct Imaging of Protein Stability and Folding Kinetics in Hydrogels. ACS Appl. Mater. Interfaces 2017, 9, 21606-21617.

(24) Kabir, A.; Dunlop, M. J.; Acharya, B.; Bissessur, R.; Ahmed, M. Water Recycling Efficacies Of Extremely Hygroscopic, Antifouling Hydrogels. RSC Adv. 2018, 8, 38100-38107.

(25) Yoshimoto, N.; Hashimoto, T.; Felix, M. M.; Umakoshi, H.; Kuboi, R. Artificial Chaperone-Assisted Refolding of Bovine Carbonic Anhydrase Using Molecular Assemblies of Stimuli-Responsive Polymers. Biomacromolecules 2003, 4, 1530-1538.

(26) Vaitheeswaran, S.; Chen, J.; Thirumalai, D. Hydrophobic and Ionic-Interactions in Bulk and Confined Water with Implications for Collapse and Folding of Proteins. J. Stat. Phys. 2011, 145, 276-292.

(27) Levy, Y.; Onuchic, N. J. Water and Proteins: A Love-Hate Relationship. Proc. Natl. Acad. Sci. U.S.A. 2004, 101, 3325-3326.

(28) Mukherjee, S.; Kar, T.; Kumar Das, P. Pyrene-Based Fluorescent Supramolecular Hydrogel: Scaffold for Energy Transfer. Chem.—Asian J. 2014, 9, 2798-2805.

(29) Gao, X.; Cao, Y.; Song, X.; Zhang, Z.; Xiao, C.; He, C.; Chen, X. pH- and Thermo-Responsive Poly(N-Isopropylacrylamide-Co-Acrylic Acid Derivative) Copolymers and Hydrogels With LCST Dependent on pH and Alkyl Side Groups. J. Mater. Chem. B 2013, 1, 5578.

(30) Belhadj, H.; Hakki, A.; Robertson, P. K. J.; Bahnemann, D. W. In situ ATR-FTIR Study of H₂O and D₂O Adsorption on TiO₂ Under UV Irradiation. Phys. Chem. Chem. Phys. 2015, 17, 22940-22946.

(31) Rostami, J.; Mathew, A. P.; Edlund, U. Zwitterionic Acetylated Cellulose Nanofibrils. Molecules 2019, 24, No. 3147.

(32) Combita, D.; Ahmed, M. Thermoresponsive and antifouling hydrogels as a radiative energy driven water harvesting system. Mat. Chem. Frontiers 2021, 5, 917-928.

(33) Diban, N.; Aguayo, T. A.; Bilbao, J.; Uritaga, A.; Ortiz, I. Membrane reactors for in situ water removal: a review of applications. Ind. Eng. Chem. Res. 2013, 52, 10342-10354.

(34) Roberts, C. J. Protein aggregation and its impact on product quality. Curr. Opin. Biotechn. 2014, 30, 211-217.

(34) Dandage, R.; Bandyopadhyay, A.; Jayaraj, G. G.; Saxena, K.; Dalal, V.; Das, A.; Chakraborty, K. Classification of chemical chaperones based on their effect on protein folding landscapes. ACS Chem. Biol. 2015, 10, 813-820.

(35) Jones, H.; Dalmaris, J.; Wright, M.; Steinke, J. H. G.; Miller, A. D. Hydrogel polymer appears to mimic the performance of the GroEL/GroES molecular chaperone machine. Org. Biomol. Chem. 2006, 4, 2568-2574.

(36) Liu, X.; Liu, Y.; Zhang, Z.; Huang, F.; Tao, Q.; Ma, R.; An, Y.; Shi, L. Temperature-responsive mixed-shell polymeric micelles for the refolding of thermally denatured proteins. Chem. Eur. J. 2013, 19, 7437-7442.

(37) Carpenter, J. F.; Pikal, M. J.; Chang, B. S.; Randolph, T. W. Rational design of stable lyophilized protein formulations some practical advice. Pharma. Res. 1997, 14, 969-975.

(38) Kabir, A.; Ahmed, M. Elucidating the role of thermally flexible hydrogels in protein refolding. ACS Appl. Biomat. 2020, https://doi.org/10.1021/acsabm.0c00324.

(39) Kabir, A.; Dunlop, M.; Acharya, B.; Bissessur, R.; Ahmed, M. Water recycling efficacies of extremely hygroscopic, antifouling hydrogels. RSC Adv. 2018, 8, 38100-38107.

(40) Schillberg, S.; Raven, N.; Spiegel, H.; Rasche, S.; Buntru, M. Critical analysis of the commercial potential of plants for the production of recombinant proteins. Front. Plant Sci. 2019, 10, 720.

(41) Pina, A. S.; Lowe, C. R.; Roque, A. C. A. Challenges and opportunities in the purification of recombinant tagged proteins. Biotechnol. Adv. 2014, 32, 366-381.

(42) Raza, F.; Zafar, H.; Zhu, Y.; Ren, Y.;-Ullah, A.; Khan, A. U.; He, X.; Han, H.; Aquib, M.; Boakye-Yiadom, K. O.; Ge, L. A review on recent advances in stabilizing peptides/proteins upon fabrication in hydrogels from biodegradable polymers. Pharmaceutics 2018, 10, 16.

(43) Roughton, B. C.; Iyer, L. K.; Bertelsen, E.; Topp, E. M.; Camarda, K. V. Protein aggregation and lyophilization: Protein structural descriptors as predictors of aggregation propensity. Comp. Chem. Eng. 2013, 58, 369-377.

(44) Roberts, C. J. Therapeutic protein aggregation: mechanisms, design, and control. Trends Biotechnol. 2014, 32, 372-380.

(45) Rajan, R. S.; Li, T.; Arakawa, T. Case studies involving protein aggregation. in aggregation of therapeutic proteins. John Wiley & Sons, Inc: Hoboken, NJ, USA, 2010; pp 367-401.

(46) Ratanji, K. D.; Derrick, J. P.; Dearman, R. J.; Kimber, I. Immunogenicity of therapeutic proteins: Influence of aggregation. J. Immunotox. 2014, 11, 99-109.

(47) Le Basle, Y.; Chennell, P.; Tokhadze, N.; Astier, A.; Sautou, V. Physicochemical stability of monoclonal antibodies: a review. J. Pharm. Sci. 2020, 109, 169-190.

(48) Roy, I.; Gupta, M. N. Freeze-drying of proteins: some emerging concerns. Biotechnol. Appl. Biochem. 2004, 39, 165-177.

(49) Lipiäinen, T.; Peltoniemi, M.; Sarkhel, S.; Yrjönen, T.; Vuorela, H.; Urtti, A.; Juppo, A. Formulation and stability of cytokine therapeutics. J. Pharm. Sci. 2015, 104, 307-326.

(50) Berraondo, P.; Sanmamed, M. F.; Ochoa, M. C.; Etxeberria, I.; Aznar, M. A.; Pérez-Gracia, J. L.; Rodríguez-Ruiz, M. E.; Ponz-Sarvise, M.; Castañón, E.; Melero, I. Cytokines in clinical cancer immunotherapy. Brit. J. Cancer 2019, 120, 6-15.

(51) Piszkiewicz, S.; Pielak, G. J. Protecting enzymes from stress-induced inactivation. Biochem. 2019, 58, 3825-3833.

(52) Babbal; Adivitiya; Mohanty, S.; Khasa, Y. P. Enzymes as therapeutic agents in human disease management. in high value fermentation products. John Wiley & Sons, Inc: Hoboken, NJ, USA, 2019; pp 225-263.

(53) Kishore, D.; Kundu, S.; Kayastha, A. M. Thermal, chemical and pH induced denaturation of a multimeric β-galactosidase reveals multiple unfolding pathways. PloS ONE 2012, 7, e50380.

(54) Vellard, M. The enzyme as drug: application of enzymes as pharmaceuticals. Curr. Opin. Biotechnol. 2003, 14, 444-450.

(55) Ahmed, M.; Pan, D.; Davis, M.E. Lack of in vivo antibody dependent cellular cytotoxicity with antibody containing gold nanoparticles. Bioconjugate Chem. 2015, 26, 812-816.

(56) Ahmed, M.; Jawanda, M.; Ishihara, K.; Narain, R. Impact of the nature, size and chain topologies of carbohydrate-phosphorylcholine polymeric gene delivery systems. Biomaterials 2012, 33, 7858-7870.

(57) Ahmed, M.; Narain, R. The effect of molecular weight, compositions and lectin type on the properties of hyperbranched glycopolymers as non-viral gene delivery systems. Biomaterials 2012, 33, 3990-4001.

(58) Ahmed, M.; Narain, R. The effect of polymer architecture, composition, and molecular weight on the properties of glycopolymer-based non-viral gene delivery systems. Biomaterials 2011, 32, 5279-5290.

(59) Global Biologics Market—Companies-to-Action Growth Insights, MD83, Healthcare, 20 Nov. 2017

All citations are hereby incorporated by reference. In the event of conflicting information with statements between any reference to or incorporated herein, and the present disclosure, the present disclosure will act as the guiding authority.

The present invention has been described with regard to one or more embodiments. However, it will be apparent to persons skilled in the art that a number of variations and modifications can be made without departing from the scope of the invention as defined in the claims.

1. A hydrogel useful for restoring a biological activity of a denatured polypeptide, wherein the hydrogel comprises a cross-linked polymer prepared by polymerization of a cross-linker and a first monomer of formula I:

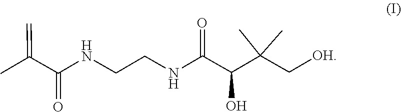 [SMILES: C=C(C)C(=O)NCCNC(=O)[C@H](O)C(C)(C)CO]

2. The hydrogel of claim 1, wherein the cross-linked polymer further comprises a second monomer.

3. The hydrogel of claim 2, wherein the second monomer is:

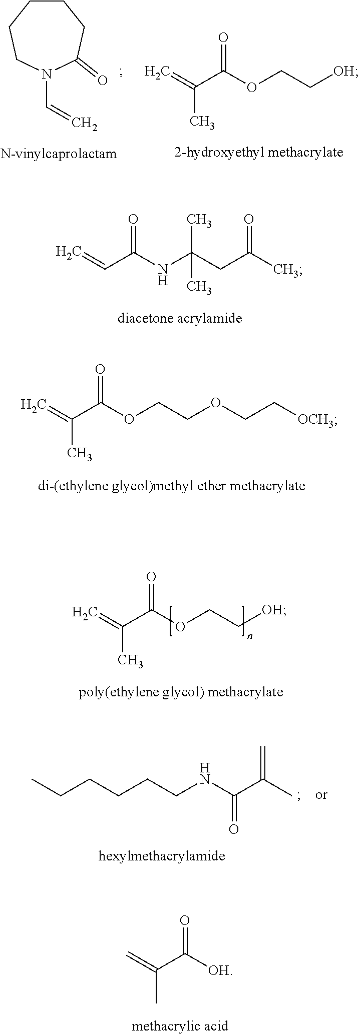

4. The hydrogel of claim 1, wherein the second monomer is:

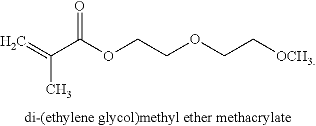

5. The hydrogel of claim 1, wherein the cross-linker is N,N′-methylenebisacrylamide (Bis).

6. The hydrogel of claim 1, wherein the hydrogel comprises about 5 mol % to about 20 mol % of cross-linker.

7. The hydrogel of claim 4, wherein the hydrogel comprises about 10 mol % of cross-linker.

8. The hydrogel of claim 1, wherein the polymerization is free radical polymerization in the presence of an initiator and a catalyst.

9. The hydrogel of claim 8, wherein the initiator is potassium persulfate (KPS), the catalyst is N,N,N′, N′-tetramethylethylenediamine (TEMEDA).

10. The hydrogel of claim 1, wherein the denatured polypeptide is denatured via heating or cooling.

11. The hydrogel of claim 10, wherein the heating comprises heating to about 110° C.

12. The hydrogel of claim 10, wherein the cooling comprises cooling to about −80° C.

13. The hydrogel of claim 1, wherein the hydrogel and the denatured polypeptide are incubated together in solution.

14. The hydrogel of claim 14, wherein the hydrogel and the denatured polypeptide are incubated together for up to about 24 hours.

15. The hydrogel of claim 15, wherein the hydrogel and the denatured polypeptide are incubated together for about 3 hours.

16. The hydrogel of claim 13, wherein the hydrogel and the denatured polypeptide are incubated together at a temperature of about 4° C. to about 50° C.

17. The hydrogel of claim 16, wherein the hydrogel and the denatured polypeptide are incubated together at a temperature of about 37° C.

18. The hydrogel of claim 1, wherein the hydrogel has a pore size of about 4 μm to about 10 μm.

19. The hydrogel of claim 16, wherein the pore size is about 8 μm.

20. The hydrogel of claim 1, wherein the denatured polypeptide is present at a concentration of up to about 2 mg per m² of hydrogel.

21. The hydrogel of claim 20, wherein the denatured polypeptide is present at a concentration of up to about 1.3 mg per m² of hydrogel.

22. The hydrogel of claim 1, wherein the denatured polypeptide is one or more of: an antibody, a cytokine, an enzyme or an antigen.

23. A method for restoring a biological activity of a denatured polypeptide, the method comprising:
combining the denatured protein with a hydrogel comprising a cross-linked polymer prepared by polymerization of a cross-linker and a first monomer of formula I: 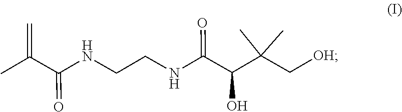 [SMILES: C=C(C)C(=O)NCCNC(=O)[C@H](O)C(C)(C)CO]
and
incubating the denatured protein and the hydrogel.

24. The method of claim 23, wherein incubating is at a temperature of about 4° C. to about 50° C.

25. The method of claim 24, wherein the temperature is about 27° C.

26. The method of 23, wherein incubating occurs over a period of about 5 min to about 24 hours.

27. The method of claim 26, wherein the period is about 3 hours.

28. A hydrogel useful for solubilizing one or more protein aggregates, wherein the hydrogel comprises a cross-linked polymer prepared by polymerization of a cross-linker and a first monomer of formula I:

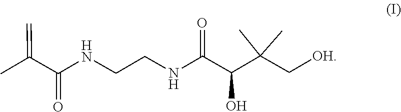 [SMILES: C=C(C)C(=O)NCCNC(=O)[C@H](O)C(C)(C)CO]

29. The hydrogel of claim 28, wherein the cross-linked polymer further comprises a second monomer.

30. The hydrogel of claim 29, wherein the second monomer is:

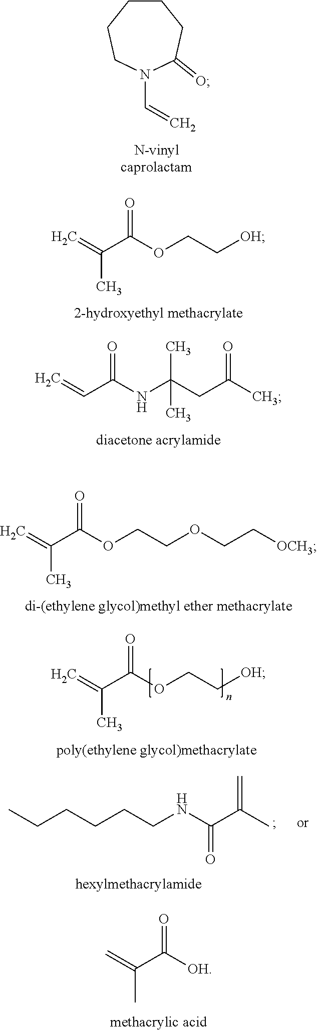

31. The hydrogel of claim 29, wherein the second monomer is:

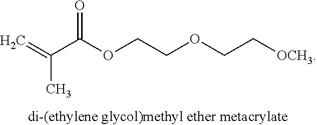

32. The hydrogel of claim 28, wherein the cross-linker is N,N′-methylenebisacrylamide (Bis).

33. The hydrogel of claim 28, wherein the hydrogel comprises about 5 mol % to about 20 mol % of cross-linker.

34. The hydrogel of claim 33, wherein the hydrogel comprises about 10 mol % of cross-linker.

35. The hydrogel of claim 28, wherein the polymerization is free radical polymerization in the presence of an initiator and a catalyst.

36. The hydrogel of claim 35, wherein the initiator is potassium persulfate (KPS), the catalyst is N,N,N′,N′-tetramethylethylenediamine (TEMEDA).

37. The hydrogel of claim 28, wherein the one or more protein aggregates comprise one or more denatured polypeptides.

38. The hydrogel of claim 37, wherein the denatured polypeptide is denatured via heating or cooling.

39. The hydrogel of claim 38, wherein the heating comprises heating to about 110° C.

40. The hydrogel of claim 38, wherein the cooling comprises cooling to about −80° C.

41. The hydrogel of claim 28, wherein the hydrogel and the one or more protein aggregates are incubated together in solution.

42. The hydrogel of claim 41, wherein the hydrogel and the one or more protein aggregates are incubated together for up to about 24 hours.

43. The hydrogel of claim 42, wherein the hydrogel and the one or more protein aggregates are incubated together for about 3 hours.

44. The hydrogel of claim 41, wherein the hydrogel and the one or more protein aggregates are incubated together at a temperature of about 4° C. to about 50° C.

45. The hydrogel of claim 44, wherein the hydrogel and the one or more protein aggregates are incubated together at a temperature of about 37° C.

46. The hydrogel of claim 28, wherein the hydrogel has a pore size of about 4 μm to about 10 μm.

47. The hydrogel of claim 46, wherein the pore size is about 8 μm.

48. The hydrogel of claim 28, wherein the one or more protein aggregates is present at a concentration of up to about 2 mg per m² of hydrogel.

49. The hydrogel of claim 48, wherein the denatured polypeptide is present at a concentration of up to about 1.3 mg per m² of hydrogel.

50. The hydrogel of claim 28, wherein the one or more protein aggregates comprises one or more of: an antibody, a cytokine, an enzyme or an antigen.

51. A method for solubilizing one or more protein aggregates, the method comprising:
combining the one or more protein aggregates with a hydrogel comprising a cross-linked polymer prepared by polymerization of a cross-linker and a first monomer of formula I: 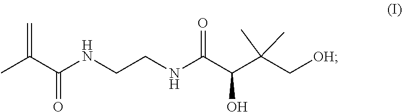 [SMILES: C=C(C)C(=O)NCCNC(=O)[C@H](O)C(C)(C)CO]
and
incubating the one or more protein aggregates and the hydrogel.

52. The method of claim 51, wherein incubating is at a temperature of about 4° C. to about 50° C.

53. The method of claim 52, wherein the temperature is about 37° C.

54. The method of claim 51, wherein incubating occurs over a period of about 5 min to about 24 hours.

55. The method of claim 54, wherein the period is about 3 hours.

56. A hydrogel useful for stabilizing a polypeptide, wherein the hydrogel comprises a cross-linked polymer prepared by polymerization of a cross-linker and a first monomer of formula I:

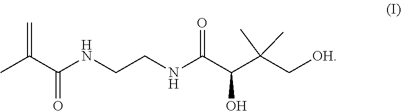 [SMILES: C=C(C)C(=O)NCCNC(=O)[C@H](O)C(C)(C)CO]

57. The hydrogel of claim 56, wherein the cross-linked polymer further comprises a second monomer.

58. The hydrogel of claim 57, wherein the second monomer is:

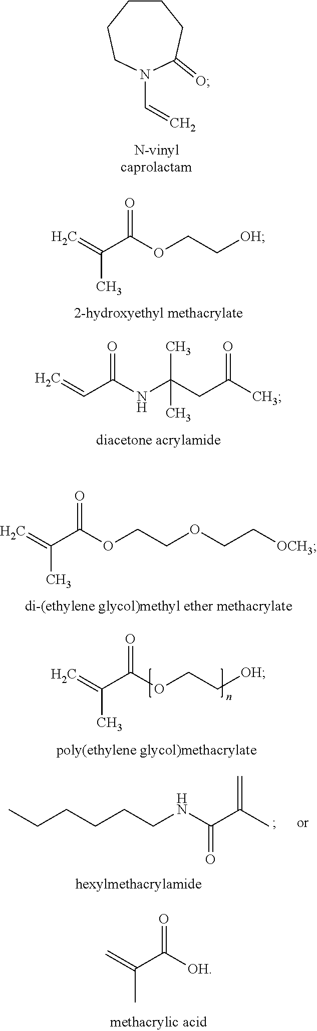

59. The hydrogel of claim 57, wherein the second monomer is:

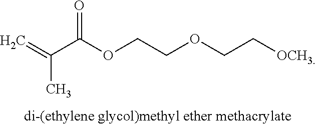

60. The hydrogel of claim 56, wherein the cross-linker is N,N′-methylenebisacrylamide (Bis).

61. The hydrogel of claim 56, wherein the hydrogel comprises about 5 mol % to about 20 mol % of cross-linker.

62. The hydrogel of claim 61, wherein the hydrogel comprises about 10 mol % of cross-linker.

63. The hydrogel of claim 56, wherein the polymerization is free radical polymerization in the presence of an initiator and a catalyst.

64. The hydrogel of claim 63, wherein the initiator is potassium persulfate (KPS), the catalyst is N,N,N′, N′-tetramethylethylenediamine (TEMEDA).

65. The hydrogel of claim 56, wherein the hydrogel and polypeptide are mixed.

66. The hydrogel of claim 65, wherein the solution is water.

67. The hydrogel of claim 65, wherein the hydrogel and polypeptide are freeze-dried.

68. The hydrogel of claim 67, wherein the hydrogel and polypeptide are reconstituted.

69. The hydrogel of claim 65, wherein the hydrogel and polypeptide are heated.

70. The hydrogel of claim 69, wherein heating comprises heating to about 110° C.

71. The hydrogel of claim 56, wherein the polypeptide is present at a concentration of up to about 2 mg per m² of hydrogel.

72. The hydrogel of claim 71, wherein the denatured polypeptide is present at a concentration of up to about 1.3 mg per m² of hydrogel.

73. The hydrogel of claim 56, wherein the one or more protein aggregates comprises one or more of: an antibody, a cytokine, an enzyme or an antigen.

74. A method of stabilizing a polypeptide, the method comprising:
combining the polypeptide with a hydrogel in a solvent, the hydrogel comprising a cross-linked polymer prepared by polymerization of a cross-linker and a first monomer of formula I: 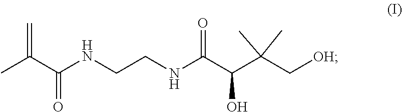 [SMILES: C=C(C)C(=O)NCCNC(=O)[C@H](O)C(C)(C)CO]
and
removing the solvent.

75. The method of claim 74, wherein the solvent is removed via freeze-drying.

76. A method of stabilizing a polypeptide, the method comprising:
combining the polypeptide with a hydrogel in a solvent, the hydrogel comprising a cross-linked polymer prepared by polymerization of a cross-linker and a first monomer of formula I: 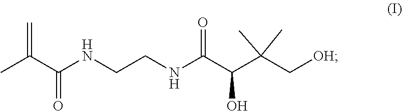 [SMILES: C=C(C)C(=O)NCCNC(=O)[C@H](O)C(C)(C)CO]
and
heating or cooling the solvent.

77. The method of claim 76, wherein heating comprises heating to about 110° C.

78. The method of claim 76, wherein the cooling comprises cooling to about −80° C.

79. A method of stabilizing a polypeptide, the method comprising:
coating one or more surfaces of a storage container for the polypeptide with a hydrogel comprising a cross-linked polymer prepared by polymerization of a cross-linker and a first monomer of formula I: 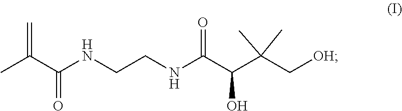 [SMILES: C=C(C)C(=O)NCCNC(=O)[C@H](O)C(C)(C)CO]
and
adding to the storage container the polypeptide.

80. The method of claim 79, wherein the coating of the one or more surfaces of the storage container comprises modifying the one or more surfaces to introduce one or more functional groups thereon that are bondable to the hydrogel.

81. The method of claim 80, wherein the coating of the one or more surfaces of the storage container comprises:
modifying the one or more surfaces to introduce a primary amine group thereon; and
modifying the one or more surfaces to introduce an alkene group thereon.

82. The method of claim 80, wherein the coating of the one or more surfaces of the storage container further comprises polymerizing the hydrogel on the one or more surfaces.

83. The method of claim 79, wherein the storage container is formed of a plastic or glass.

84. The method of claim 79, wherein the polypeptide is a laboratory-grade polypeptide.

85. The method of claim 79, wherein the polypeptide is the component of a vaccine.
